(19) United States
(12) Patent Application Publication
(10) Pub. No.: US 2004/0248979 A1
(43) Pub. Date: Dec. 9, 2004

(54) Method of treating lower urinary tract disorders

(75) Inventors: Lee R. Brettman, Sudbury, MA (US); Steven B. Landau, Wellesley, MA (US); Matthew O. Fraser, Apex, NC (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DYNOGEN PHARMACEUTICALS, INC., BOSTON, MA

(21) Appl. No.: 10/859,922

(22) Filed: Jun. 3, 2004

Related U.S. Application Data
(60) Provisional application No. 60/475,636, filed on Jun. 3, 2003.

(51) Int. Cl. A61K 31/195; A61K 31/137

(52) U.S. Cl. 514/561; 514/650

(57) ABSTRACT

The invention relates to a method of treating at least one symptom of a lower urinary tract disorder in a subject in need of treatment wherein the symptom is selected from the group consisting of urinary frequency, urinary urgency, urinary urge incontinence, nocturia and enuresis comprising coadministering to said subject a first amount of an α₂δ subunit calcium channel ligand and a second amount of a substituted aminomethyl-phenyl-cyclohexane derivative, wherein the first and second amounts together comprise a therapeutically effective amount. The coadministration of a first amount of an α₂δ subunit calcium channel ligand and a second amount of a substituted aminomethyl-phenyl-cyclohexane derivative can result in an enhanced or synergistic therapeutic effect, wherein the combined effect is greater than the additive effect resulting from separate administration of the first amount of the α₂δ subunit calcium channel ligand and the second amount of the substituted aminomethyl-phenyl-cyclohexane derivative.

### RELATED APPLICATION

This application claims the benefit of U.S. Provisional Application No. 60/475,636, filed Jun. 3, 2003.

The entire teachings of the above application are incorporated herein by reference.

### BACKGROUND OF THE INVENTION

Lower urinary tract disorders affect the quality of life of millions of men and women in the United States every year. While the kidneys filter blood and produce urine, the lower urinary tract functions to store and periodically eliminate urine and includes all other parts of the urinary tract except the kidneys. Generally, the lower urinary tract includes the ureters, the urinary bladder, sphincter and the urethra. Disorders of the lower urinary tract include overactive bladder, interstitial cystitis, prostatitis, prostadynia and benign prostatic hyperplasia.

Overactive bladder is a treatable medical condition that is estimated to affect 17 to 20 million people in the United States. Symptoms of overactive bladder can include urinary frequency, urinary urgency, urinary urge incontinence (accidental loss of urine) due to a sudden and unstoppable need to urinate, nocturia (the disturbance of sleep because of the need to urinate) or enuresis resulting from overactivity of the detrusor muscle (the smooth muscle of the bladder which contracts and causes it to empty).

Neurogenic overactive bladder (or neurogenic bladder) is a type of overactive bladder which occurs as a result of detrusor muscle overactivity referred to as detrusor hyperreflexia, secondary to known neurologic disorders. Patients with neurologic disorders, such as stroke, Parkinson's disease, diabetes, multiple sclerosis, peripheral neuropathy, or spinal cord lesions often suffer from neurogenic overactive bladder. In contrast, non-neurogenic overactive bladder occurs as a result of detrusor muscle overactivity referred to as detrusor muscle instability. Detrusor muscle instability can arise from non-neurological abnormalities, such as bladder stones, muscle disease, urinary tract infection or drug side effects or can be idiopathic.

Due to the enormous complexity of micturition (the act of urination) an exact mechanism which causes overactive bladder is not known. Overactive bladder can result from hypersensitivity of sensory neurons of the urinary bladder, arising from various factors including inflammatory conditions, hormonal imbalances, and prostate hypertrophy. Destruction of the sensory nerve fibers, either from a crushing injury to the sacral region of the spinal cord, or from a disease that causes damage to the dorsal root fibers as they enter the spinal cord can also lead to overactive bladder. In addition, damage to the spinal cord or brain stem causing interruption of transmitted signals can lead to abnormalities in micturition. Therefore, both peripheral and central mechanisms can be involved in mediating the altered activity in overactive bladder.

In spite of the uncertainty regarding whether central or peripheral mechanisms, or both, are involved in overactive bladder, many proposed mechanisms implicate neurons and pathways that mediate non-painful visceral sensation. Somatosensory information from the bladder is relayed by nociceptive Aδ and C fibers that enter the spinal cord via the dorsal root ganglion (DRG) and project to the brainstem and thalamus via second or third order neurons (Andersson (2002) Urology 59:18-24; Andersson (2002) Urology 59:43-50; Morrison, J., Steers, W. D., Brading, A., Blok, B., Fry, C., de Groat, W. C., Kakizaki, H., Levin, R., and Thor, K. B., “Basic Urological Sciences,” In: Incontinence (vol. 2) Abrams, P. Khoury, S., and Wein, A. (Eds.) Health Publications, Ltd., Plymbridge Distributors, Ltd., Plymouth, UK., (2002)). A number of different subtypes of sensory afferent neurons can be involved in neurotransmission from the lower urinary tract. These can be classified as, but not limited to, small diameter, medium diameter, large diameter, myelinated, unmyelinated, sacral, lumbar, peptidergic, non-peptidergic, IB4 positive, IB4 negative, C fiber, Aδ fiber, high threshold or low threshold neurons. Nociceptive input to the DRG is thought to be conveyed to the brain along several ascending pathways, including the spinothalamic, spinoreticular, spinomesencephalic, spinocervical, and in some cases dorsal column/medial lemniscal tracts (A. I. Basbaum and T. M. Jessell (2000) “The perception of pain,” In Principles of Neural Science, 4th. ed.).

Current treatments for overactive bladder include medication, diet modification, programs in bladder training, electrical stimulation, and surgery. Currently, antimuscarinics (which are members of the general class of anticholinergics) are the primary medication used for the treatment of overactive bladder. The antimuscarinic, oxybutynin, has been the mainstay of treatment for overactive bladder. However, treatment with antimuscarinics suffers from limited efficacy and side effects such as dry mouth, dry eyes, dry vagina, blurred vision, cardiac side effects, such as palpitations and arrhythmia, drowsiness, urinary retention, weight gain, hypertension and constipation, which have proven difficult for some individuals to tolerate. Currently there are no clinically approved applications of central nervous system oriented pharmacotherapies for treating lower urinary tract disorders, such as overactive bladder.

Interstitial cystitis is another lower urinary tract disorder of unknown etiology that predominantly affects young and middle-aged females, although men and children can also be affected. Symptoms of interstitial cystitis can include irritative voiding symptoms, urinary frequency, urinary urgency, nocturia or suprapubic or pelvic pain related to and relieved by voiding. Many interstitial cystitis patients also experience headaches as well as gastrointestinal and skin problems. In some cases, interstitial cystitis can also be associated with ulcers or scars of the bladder. (Metts, J. F. (2001) Interstitial Cystitis: Urgency and Frequency Syndrome. American Family Physician 64(7): 1199-1206).

Currently, the only FDA-approved oral medication for use in interstitial cystitis is ELMIRON® (pentosan polysulfate sodium). ELMIRON® was approved in 1996 and is thought to work by restoring a damaged, thin or leaky bladder surface. However, ELMIRON® must be taken continually for several months before any improvements can be expected. As such, lack of patient compliance often results in unsuccessful treatment. In addition, treatment with ELMIRON® is not effective in a large percentage of patients.

Other medications which have been used “off-label” for the treatment of interstitial cystitis include, for example, antidepressants, antihistamines and anticonvulsants (See, Theoharides, T. C. et al. “New agents for the medical treatment of interstitial cystitis,” Exp. Opin. Invest. Drugs 10(3): 521-46 (2001)). However, in view of the unknown cause of interstitial cystitis and the suggestion that the disorder is multifactorial in origin, these additional therapies have not provided adequate relief of the associated symptoms.

Prostatitis and prostadynia are other lower urinary tract disorders that have been suggested to affect approximately 2-9% of the adult male population (Collins, M. M. et al., (1998) “How common is prostatitis? A national survey of physician visits,” Journal of Urology, 159: 1224-1228). Prostatitis is an inflammation of the prostate, and includes bacterial prostatitis (acute and chronic) and non-bacterial prostatitis. Acute and chronic bacterial prostatitis are characterized by inflammation of the prostate and bacterial infection of the prostate gland, usually associated with symptoms of pain, urinary frequency and/or urinary urgency. Chronic bacterial prostatitis is distinguished from acute bacterial prostatitis based on the recurrent nature of the disorder. Chronic non-bacterial prostatitis is characterized by inflammation of the prostate which is of unknown etiology accompanied by the presence of an excessive amount of inflammatory cells in prostatic secretions not currently associated with bacterial infection of the prostate gland, and usually associated with symptoms of pain, urinary frequency and/or urinary urgency. Prostadynia is a disorder which mimics the symptoms of prostatitis absent inflammation of the prostate, bacterial infection of the prostate and elevated levels inflammatory cells in prostatic secretions. Prostadynia can be associated with symptoms of pain, urinary frequency and/or urinary urgency.

Currently, there are no established treatments for prostatitis and prostadynia. Antibiotics are often prescribed, but with little evidence of efficacy. COX-2 selective inhibitors and α-adrenergic blockers and have been suggested as treatments, but their efficacy has not been established. Hot sitz baths and anticholinergic drugs have also been employed to provide some symptomatic relief.

Benign prostatic hyperplasia (BPH) is a non-malignant enlargement of the prostate that is very common in men over 40 years of age. BPH is thought to be due to excessive cellular growth of both glandular and stromal elements of the prostate. Symptoms of BPH can include urinary frequency, urinary urgency, urge incontinence, nocturia, or reduced urinary force and speed of flow.

Invasive treatments for BPH include transurethral resection of the prostate, transurethral incision of the prostate, balloon dilation of the prostate, prostatic stents, microwave therapy, laser prostatectomy, transrectal high-intensity focused ultrasound therapy and transurethral needle ablation of the prostate. However, complications can arise through the use of some of these treatments, including retrograde ejaculation, impotence, postoperative urinary tract infection and some urinary incontinence. Non-invasive treatments for BPH include androgen deprivation therapy and the use of 5α-reductase inhibitors and α-adrenergic blockers. However, these treatments have proven only minimally to moderately effective for some patients.

In view of the limitations associated with existing therapies and treatments for lower urinary tract disorders, new therapies and treatments are highly desirable.

### SUMMARY OF THE INVENTION

The invention relates to a method of treating at least one symptom of a lower urinary tract disorder in a subject in need of treatment wherein the symptom is selected from the group consisting of urinary frequency, urinary urgency, urinary urge incontinence, nocturia and enuresis comprising coadministering to said subject a first amount of an α₂δ subunit calcium channel ligand and a second amount of a substituted aminomethyl-phenyl-cyclohexane derivative, wherein the first and second amounts together comprise a therapeutically effective amount.

In one embodiment, coadministration of a first amount of an α₂δ subunit calcium channel ligand and a second amount of a substituted aminomethyl-phenyl-cyclohexane derivative can result in an enhanced or synergistic therapeutic effect, wherein the combined effect is greater than the additive effect resulting from separate administration of the first amount of the α₂δ subunit calcium channel ligand and the second amount of the substituted aminomethyl-phenyl-cyclohexane derivative.

In one embodiment, the lower urinary tract disorder can be selected from the group consisting of overactive bladder, interstitial cystitis, prostatitis, prostadynia and benign prostatic hyperplasia.

In another embodiment, the lower urinary tract disorder is overactive bladder.

In yet another embodiment, the lower urinary tract disorder is interstitial cystitis.

The invention further relates to pharmaceutical compositions useful for the treatment of at least one symptom of a lower urinary tract disorder in a subject in need of treatment wherein the symptom is selected from the group consisting of urinary frequency, urinary urgency, urinary urge incontinence, nocturia and enuresis. The pharmaceutical composition comprises a first amount of an α₂δ subunit calcium channel ligand and a second amount of a substituted aminomethyl-phenyl-cyclohexane derivative. The pharmaceutical compositions of the present invention can optionally contain a pharmaceutically acceptable carrier. The first amount of an α₂δ subunit calcium channel ligand and the second amount of a substituted aminomethyl-phenyl-cyclohexane derivative can together comprise a therapeutically effective amount.

In one embodiment, the lower urinary tract disorder treated with a pharmaceutical composition can be selected from the group consisting of overactive bladder, interstitial cystitis, prostatitis, prostadynia and benign prostatic hyperplasia.

In another embodiment, the lower urinary tract disorder is overactive bladder.

In yet another embodiment, the lower urinary tract disorder is interstitial cystitis.

The invention further relates to the use of a pharmaceutical composition comprising a first amount of an α₂δ subunit calcium channel ligand and a second amount of a substituted aminomethyl-phenyl-cyclohexane derivative for the manufacture of a medicament for the treatment of at least one symptom of a lower urinary tract disorder in a subject in need of treatment wherein the symptom is selected from the group consisting of urinary frequency, urinary urgency, urinary urge incontinence, nocturia and enuresis. The pharmaceutical composition used for the manufacture of a medicament can optionally contain a pharmaceutically acceptable carrier. The first amount of an α₂δ subunit calcium channel ligand and the second amount of a substituted aminomethyl-phenyl-cyclohexane derivative can together comprise a therapeutically effective amount.

The method of coadministration of a first amount of an α₂δ subunit calcium channel ligand and a second amount of a substituted aminomethyl-phenyl-cyclohexane derivative can result in an enhanced or synergistic therapeutic effect, wherein the combined effect is greater than the additive effect that would result from separate administration of the first amount of the α₂δ subunit calcium channel ligand and the second amount of the substituted aminomethyl-phenyl-cyclohexane derivative. An advantage of the synergistic effect of the combination therapy is the ability to use less of each agent than is needed when each is administered alone. As such, undesirable side effects associated with the agents are reduced (partially or completely). A reduction in side effects can result in increased patient compliance over current treatments.

The foregoing and other objects, features and advantages of the invention will be apparent from the following more particular description of preferred embodiments of the invention.

### BRIEF DESCRIPTION OF THE DRAWINGS

FIG. 1 is a graph of bladder capacity in mL versus the effect of cumulative increasing doses of tramadol (n=4), gabapentin (n=11) and their matched combinations (Low dose for the combination was 30 mg/kg gabapentin and 3 mg/kg tramadol; n=6) in rats subjected to the dilute acetic acid model described herein.

FIG. 2 is a graph of % Recovery from Irritation (bladder capacity in mL normalized) versus the effects of increasing doses of gabapentin (n=11), tramadol (n=4) and their matched dose combinations in rats subjected to the dilute acetic acid model described herein.

FIG. 3 is a graph of % Recovery from Irritation (bladder capacity in mL, normalized) versus the theoretical additive effects of increasing doses of gabapentin and tramadol and the effects of increasing dose combinations of gabapentin and tramadol, in rats subjected to the dilute acetic acid model described herein.

### DETAILED DESCRIPTION OF THE INVENTION

The invention relates to a method of treating at least one symptom of a lower urinary tract disorder in a subject in need of treatment wherein the symptom is selected from the group consisting of urinary frequency, urinary urgency, urinary urge incontinence, nocturia and enuresis. In one embodiment, the lower urinary tract disorder can be selected from the group consisting of overactive bladder, interstitial cystitis, prostatitis, prostadynia and benign prostatic hyperplasia. In another embodiment, the lower urinary tract disorder is overactive bladder. In yet another embodiment, the lower urinary tract disorder is interstitial cystitis.

### α₂δ Subunit Calcium Channel Ligand

Voltage-gated calcium channels are heteromultimers composed of an α₁ subunit and three auxiliary subunits, α₂δ, β and γ. The α₁ subunit forms the ion pore and possesses gating functions and, in some cases, drug binding sites. The current through the α₁ subunit is modulated by interactions with the α₂δ, β and γ subunits. There are three families of α₁ subunits: L-type, Caᵥ1 family, composed of α_{1C} (cardiac, Caᵥ1.2), α_{1D} (neuronal/endocrine), α_{1S} (skeletal muscle), and α_{1F} (retinal) subunits; the non-L-type high voltage-activated, or Caᵥ2 family, which contains P- and Q-types encoded by α_{1A} subunits; the N-type encoded by α_{1B} subunits (Caᵥ2.2); R-types encoded by α_{1E}; and the T-type family, or Caᵥ3 family, encoded by α_{1G} (Caᵥ3.1), α_{1H}, and α₁₁ subunits. The α₁ subunits each have four homologous domains (I-IV) that are each composed of six transmembrane helices. The fourth transmembrane helix of each domain contains the voltage-sensing function. The four a, domains cluster in the membrane to form the ion pore. The β-subunit is localized intracellularly and is involved in the membrane trafficking of α₁ subunits. The γ-subunit is a glycoprotein having four transmembrane segments. The α₂ subunit is a highly glycosylated extracellular protein that is attached to the membrane-spanning δ-subunit by means of disulfide bonds (an α₂δ subunit). The α₂-domain provides structural support required for channel stimulation, while the δ domain modulates the voltage-dependent activation and steady-state inactivation of the channel.

As used herein, α₂δ subunit of a calcium channel refers to naturally occurring α₂δ subunits of a calcium channel (e.g., mammalian α₂δ subunits of a calcium channel (e.g., human (Homo sapiens) α₂δ subunits of a calcium channel, murine (e.g., rat, mouse) α₂δ subunits of a calcium channel)) and to proteins having an amino acid sequence which is the same as that of a corresponding naturally occurring α₂δ subunit of a calcium channel (e.g., recombinant proteins). The term includes naturally occurring variants, such as polymorphic or allelic variants and splice variants. Several genes encoding α₂δ subunits have been identified (e.g., α₂δ-1, α₂δ-2, α₂δ-3 and α₂δ-4: See, Qin, N. et al., Mol. Pharmacol. 62(3): 485-496 (2002); Marais, E. et al., Mol. Pharmacol. 59(5): 1243-1248 (2001); Klugbauer, N. et al., J. Neurosci. 19: 684-691(1999); Brown, J. P. et al., J. Biol. Chem. 273(39): 25458-25465 (1998); DeJongh, K. S. et al., J. Biol. Chem. 265(25): 14738-14741 (1990); Ellis, S. B. et al., Science 241: 1661-1664 (1988); and U.S. Pat. No. 6,441,156 B1 to Lerman et al.).

The term α₂δ subunit calcium channel ligand, as used herein refers to a substance which interacts with (e.g., binds to) an α₂δ subunit of a calcium channel. In one embodiment, ligand binding of an α₂δ subunit of a calcium channel occurs with high affinity. The α₂δ subunit calcium channel ligand includes, but is not limited to, a natural ligand, whether isolated, purified, synthetic, and/or recombinant, a homolog of a natural ligand (e.g., from another mammal), antibodies, portions of such molecules and other substances which bind an α₂δ subunit calcium channel. It is preferred that the α₂δ subunit calcium channel ligand is other than a natural ligand. The term α₂δ subunit calcium channel ligand encompasses substances which are antagonists or agonists of the activity of an α₂δ subunit of a calcium channel, as well as substances which selectively bind an α₂δ subunit of a calcium channel, but lack antagonist or agonist activity.

As used herein, an antagonist of an α₂δ subunit of a calcium channel is a substance which inhibits at least one function characteristic of an α₂δ subunit of a calcium channel, such as a binding activity or modulation of calcium channel activity.

As used herein, an agonist of an α₂δ subunit of a calcium channel is a substance which promotes (induces or enhances) at least one function characteristic of an α₂δ subunit of a calcium channel, such as binding activity or modulation of calcium channel activity.

Suitable methods for determining the binding affinity of a compound for the α₂δ subunit of calcium channels can be found in, for example, Gee et al., J. Biol. Chem. 271:5768-5776 (1996) and U.S. Pat. No. 6,441,156 B1, which are incorporated herein by reference.

Suitable α₂δ subunit calcium channel ligands include any compound that binds to an α₂δ subunit of a calcium channel as disclosed further herein, for example, GABA (gamma-aminobutyric acid) analogs such as gabapentin and pregabalin and the salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

GABA analogs are compounds that are derived from or based on gamma-aminobutyric acid. GABA analogs are either readily available or can be readily synthesized using known methods. Exemplary GABA analogs and their salts include gabapentin and pregabalin, and other GABA analogs as described in U.S. Pat. No. 4,024,175, U.S. Pat. No. 5,563,175, U.S. Pat. No. 6,316,638, U.S. Pat. No. 6,545,022 B1, PCT Publication No. WO 93/23383, UK Patent Application GB 2 374 595, Bryans et al., J. Med. Chem. 41:1838-1845 (1998), and Bryans et al., Med. Res. Rev. 19:149-177 (1999), which are incorporated herein by reference.

Other α₂δ subunit calcium channel ligands useful in the practice of the invention, include those disclosed in U.S. Application No. 20020111338, cyclic amino acid compounds as disclosed in PCT Publication No. WO 99/08670, compositions disclosed in PCT Publication No. WO 99/08670, U.S. Pat. No. 6,342,529, controlled release formulations as disclosed in U.S. Application No. 20020119197 and U.S. Pat. No. 5,955,103, and sustained release compounds and formulations as disclosed in PCT Publication No. WO 02/28411, PCT Publication No. WO 02/28881, PCT Publication No. WO 02/28883, PCT Publication No. WO 02/32376, PCT Publication No. WO 02/42414, U.S. Application No. 20020107208, U.S. Application No. 20020151529, and U.S. Application No. 20020098999, which are incorporated herein by reference.

Gabapentin (NEURONTIN® or 1-(aminomethyl) cyclohexaneacetic acid) is an anticonvulsant drug with a high binding affinity for certain calcium channel subunits. Although gabapentin was originally developed as a GABA-mimetic compound to treat spasticity, gabapentin has no direct GABAergic action and does not block GABA uptake or metabolism. (For review, see Rose et al. (2002) Analgesia 57:451-462). However, gabapentin has been found to be an effective treatment for the prevention of partial seizures in patients who are refractory to other anticonvulsant agents (Chadwick (1991) “Gabapentin,” In Recent Advances in Epilepsy, Pedley T A, Meldrum B S (eds.), Churchill Livingstone, N.Y., pp. 211-222). Gabapentin and the related drug pregabalin interact with α₂δ subunits of calcium channels (Gee et al. (1996) J. Biol. Chem. 271: 5768-5776 and Marais, E. et al., Mol. Pharmacol. 59(5): 1243-1248 (2001)).

In addition to its known anticonvulsant effects, gabapentin has been shown to block the tonic phase of nociception induced by formalin and carrageenan, and exerts an inhibitory effect in neuropathic pain models of mechanical hyperalgesia and mechanical/thermal allodynia (Rose et al., Analgesia 57: 451-462 (2002)). Double-blind, placebo-controlled trials have indicated that gabapentin is an effective treatment for painful symptoms associated with diabetic peripheral neuropathy, post-herpetic neuralgia, and neuropathic pain (see, e.g., Backonja et al., JAMA 280:1831-1836 (1998); Mellegers et al., Clin. J. Pain 17:284-95 (2001)).

Pregabalin, (3S)-3-(aminomethyl)-5-methylhexanoic acid or (S)-(+)-3-isobutyl GABA (Chemical Abstracts Registry No. 148553-50-8) is another GABA analog the use of which as an anticonvulsant has been explored (Bryans et al., J. Med. Chem. 41:1838-1845 (1998)). Pregabalin has been shown to possess even higher binding affinity for certain α2δ subunits of calcium channels than gabapentin (Bryans et al. Med. Res. Rev. 19:149-177 (1999)).

Other GABA analogs which display binding affinity to the α₂δ subunits of calcium channels include, but are not limited to, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, and (7-(aminomethyl)bicyclo[2.2.1]hept-7-yl)acetic acid (Bryans et al., J. Med. Chem. 41:1838-1845 (1998); Bryans et al., Med. Res. Rev. 19:149-177 (1999)).

Additional α₂δ subunit calcium channel ligands suitable for use in the present invention include those described in U.S. Pat. No. 6,492,375, U.S. Pat. No. 6,294,533, U.S. Pat. No. 6,011,035, U.S. Pat. No. 6,387,897, U.S. Pat. No. 6,310,059, U.S. Pat. No. 6,267,945, PCT Publication No. WO 01/49670, PCT Publication No. WO 01/46166, and PCT Publication No. WO 01/45709, which are incorporated herein by reference.

Exemplary GABA analogs and fused bicyclic or tricyclic amino acid analogs of gabapentin that are useful in the present invention include:

1. Gabapentin or salts, enantiomers, analogs, esters, amides, prodrugs, active metabolites, or derivatives thereof;

2. Pregabalin or salts, enantiomers, analogs, esters, amides, prodrugs, active metabolites, or derivatives thereof;

3. GABA analogs according to the following structure as described in U.S. Pat. No. 4,024,175, or salts, enantiomers, analogs, esters, amides, prodrugs, active metabolites, or derivatives thereof,

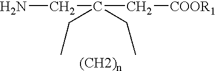

wherein R₁ is hydrogen or a lower alkyl radical and n is 4, 5, or 6;

4. GABA analogs according to the following structure as described in U.S. Pat. No. 5,563,175, or salts, enantiomers, analogs, esters, amides, prodrugs, active metabolites, or derivatives thereof,

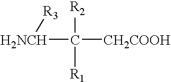

wherein R₁ is a straight or branched alkyl group having from 1 to 6 carbon atoms, phenyl, or cycloalkyl having from 3 to 6 carbon atoms; R₂ is hydrogen or methyl; and R₃ is hydrogen, methyl or carboxyl;

5. Substituted amino acids according to the following structures as described in U.S. Pat. No. 6,316,638, or salts, enantiomers, analogs, esters, amides, prodrugs, active metabolites, or derivatives thereof,

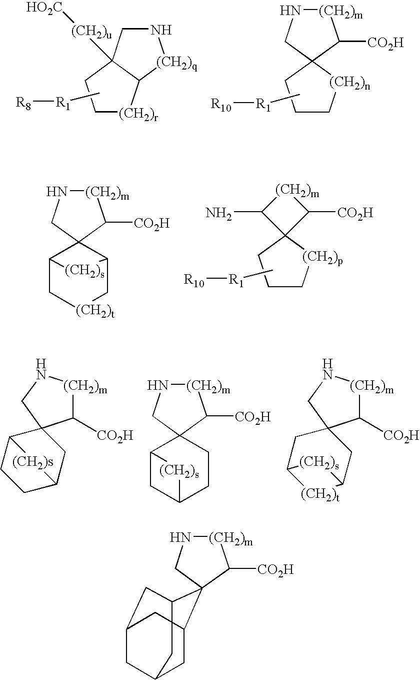

wherein R₁ to R₁₀ are each independently selected from hydrogen or a straight or branched alkyl of from 1 to 6 carbons, benzyl, or phenyl; m is an integer of from 0 to 3; n is an integer from 1 to 2; p is an integer from 1 to 2; q is an integer from 0 to 2; r is an integer from 1 to 2; s is an integer from 1 to 3; t is an integer from 0 to 2; and u is an integer from 0 to 1;

6. GABA analogs as disclosed in PCT Publication No. WO 93/23383 or salts, enantiomers, analogs, esters, amides, prodrugs, active metabolites, or derivatives thereof;

7. GABA analogs as disclosed in Bryans et al. (1998) J. Med. Chem. 41:1838-1845 or salts, enantiomers, analogs, esters, amides, prodrugs, active metabolites, or derivatives thereof;

8. GABA analogs as disclosed in Bryans et al. (1999) Med. Res. Rev. 19:149-177 or salts, enantiomers, analogs, esters, amides, prodrugs, active metabolites, or derivatives thereof;

9. Amino acid compounds according to the following structure as described in U.S. Application No. 20020111338, or salts, enantiomers, analogs, esters, amides, prodrugs, active metabolites, or derivatives thereof;

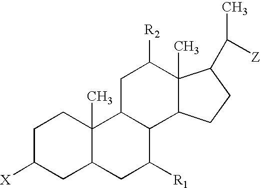

wherein R₁ and R₂ are independently hydrogen or hydroxy; X is selected from the group consisting of hydroxy and Q^{x}-G- where:

G is —O—, —C(O)O— or —NH—;

Q^{x} is a group derived from a linear oligopeptide comprising a first moiety D and further comprising from 1 to 3 amino acids, and wherein said group is cleavable from the amino acid compound under physiological conditions;

D is a GABA analog moiety;

Z is selected from the group consisting of:

(i) a substituted alkyl group containing a moiety which is negatively charged at physiological pH, which moiety is selected from the group consisting of —COOH, —SO₃H, —SO₂H, —P(O)(OR¹⁶)(OH), —OP(O)(OR¹⁶)(OH), —OSO₃H and the like, and where R¹⁶ is selected from the group consisting of alkyl, substituted alkyl, aryl and substituted aryl; and

(ii) a group of the formula -M-Q^{x′}, wherein M is selected from the group consisting of —CH₂OC(O)— and —CH₂CH₂C(O)—, and wherein Q^{x′} is a group derived from a linear oligopeptide comprising a first moiety D′ and further comprising from 1 to 3 amino acids, and wherein said group is cleavable under physiological conditions; D′ is a GABA analog moiety; or a pharmaceutically acceptable salt thereof; provided that when X is hydroxy, then Z is a group of formula -M-Q^{x′};

10. Cyclic amino acid compounds as disclosed in PCT Publication No. WO 99/08670 or salts, enantiomers, analogs, esters, amides, prodrugs, active metabolites, or derivatives thereof;

11. Cyclic amino acids according to the following structures as disclosed in PCT Publication No. W099/21824, or salts, enantiomers, analogs, esters, amides, prodrugs, active metabolites, or derivatives thereof,

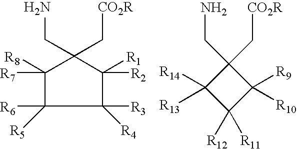

wherein R is hydrogen or a lower alkyl; R₁ to R₁₄ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R₁₅, —CH₂CO₂H, —CHCO₂R₁₅, —OR₁₅ wherein R₁₅ is a straight or branched alkyl of from 1 to 6 carbons, phenyl, or benzyl, and R₁ to R₈ are not simultaneously hydrogen;

12. Bicyclic amino acids according to the following structures wherein n is an integer as disclosed in U.S. patent application Ser. No. 60/160725, including those disclosed as having high activity as measured in a radioligand binding assay using [3H]gabapentin and the α2δ subunit derived from porcine brain tissue, or acids, salts, enantiomers, analogs, esters, amides, prodrugs, active metabolites, and derivatives thereof,

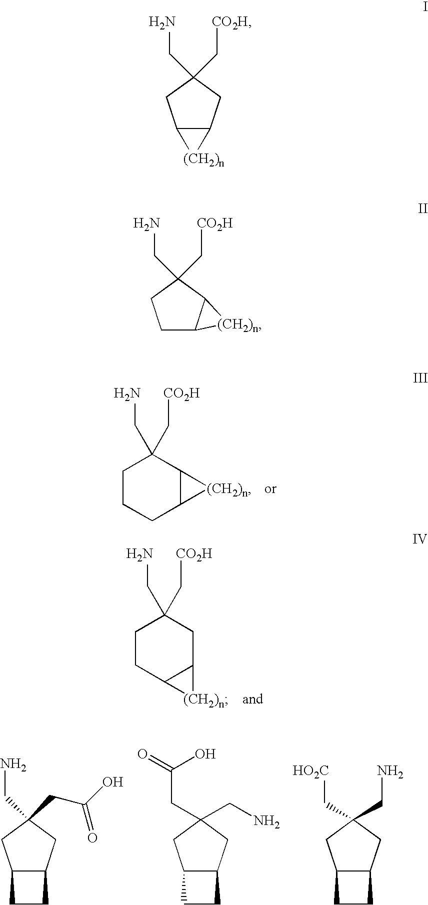

13. Bicyclic amino acid analogs according to the following structures as disclosed in UK Patent Application GB 2 374 595 and acids, salts, enantiomers, analogs, esters, amides, prodrugs, active metabolites, and derivatives thereof,

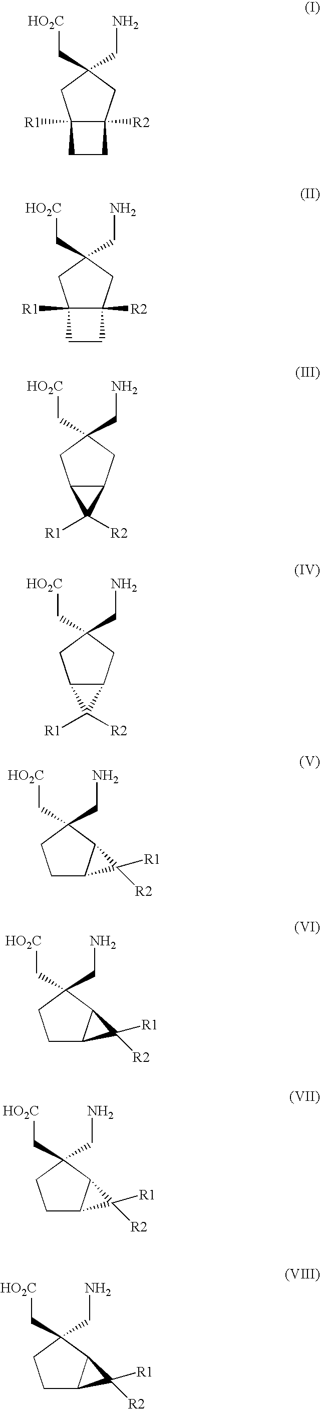

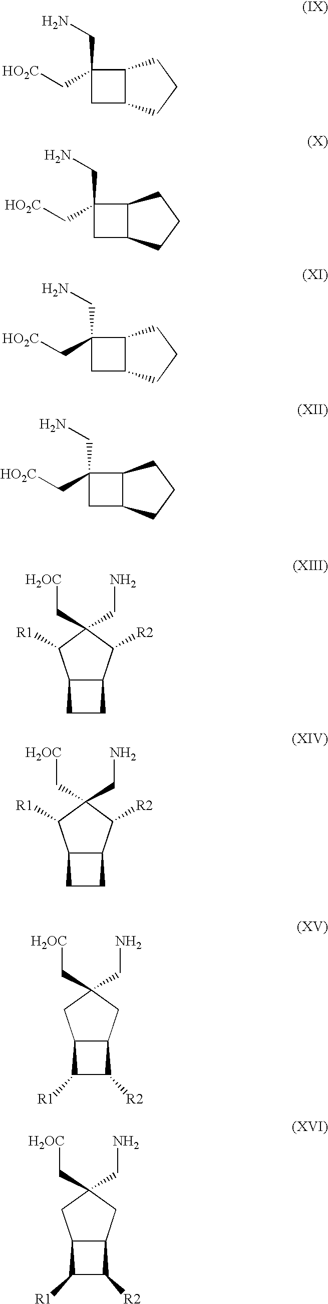

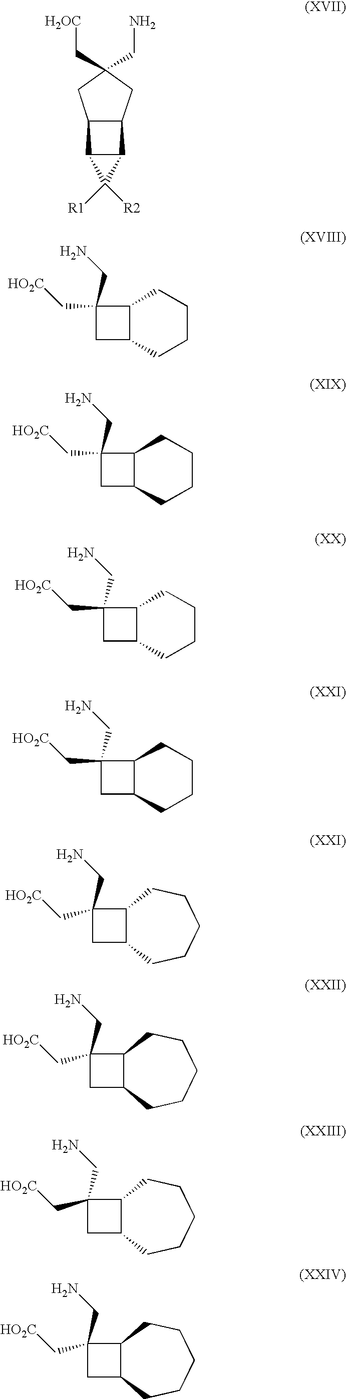

wherein R₁ and R₂ are independently selected from H, straight or branched alkyl of 1-6 carbon atoms, cycloalkyl of from 3-6 carbons atoms, phenyl and benzyl, subject to the proviso that, except in the case of a tricyclooctane compound of formula (XVII), R1 and R2 are not simultaneously hyrogen.

### Substituted Aminomethyl-Phenyl-Cyclohexane Derivatives

The substituted aminomethyl-phenyl-cyclohexane derivatives suitable for use in the invention are represented by structural Formula I:

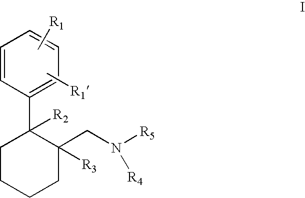

and enantiomers and mixtures thereof wherein:

R₁ and R₁′ are independently hydrogen, an aliphatic group, an aryl group, an arylalkyl group, a halogen, —CN, —OR₆, —SR₆, —NR₆R₆, —OC(O)R₆, —C(O)OR₆, —C(O)R₆ or —C(O)NR₆R₆;

R₂ is hydrogen, halogen, —OR₇ or —OC(O)R₇;

R₃ is hydrogen or an aliphatic group;

or R₂ and R₃ together form a double bond;

R₄ and R₅ are independently hydrogen, an aliphatic group, an aryl group or an arylalkyl group;

R₆ is hydrogen, an aliphatic group, an aryl group or an arylalkyl group;

R₇ is hydrogen, an aliphatic group, an aryl group or an arylalkyl group;

or pharmaceutically acceptable salts, solvates or hydrates thereof.

In a particular embodiment of Formula I, R₂ is —OH. When R₂ is —OH, it is preferred that R₁′ is hydrogen and R₁ is —OCH₃, preferably substituted at the meta position of the phenyl ring.

In a further embodiment of Formula I, R₂ is —OH, R₁′ is hydrogen and R₁ is —OR₆, substituted at the meta position of the phenyl ring and R₆ is an aliphatic group, for example, an alkyl group. In a particular embodiment, wherein R₂ is —OH, R₁′ is hydrogen and R₁ is —OR₆, substituted at the meta position of the phenyl ring and R₆ is an alkyl group, R₃, R₄ and R₅ can be hydrogen or an alkyl group.

In one embodiment, the substituted aminomethyl-phenyl-cyclohexane derivative suitable for use in the invention is represented by structural Formula II:

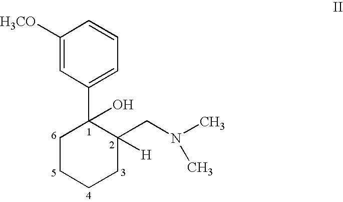

and enantiomers and mixtures thereof or pharmaceutically acceptable salts, solvates or hydrates thereof.

In a particular embodiment, the compound of Formula II is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula II is a 50:50 mixture of (+)cis and (−)cis enantiomers as shown below:

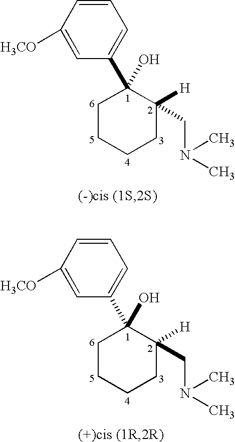

In other words, the compound of Formula II is the 50:50 mixture of (+/−)cis-2-[(dimethylamino)methyl]-1-(3-methoxyphenyl) cyclohexanol, commonly referred to as tramadol. The compound can be in the form of a pharmaceutically acceptable salt. Typically, tramadol is administered in the form of the hydrochloride salt. The tramadol hydrochloride is also known, for example, by the tradename ULTRAM®.

Tramadol in the form of the hydrochloride salt, is widely used as an analgesic. Tramadol is a centrally acting analgesic with a low affinity for opioid receptors. In contrast to other opioids, the analgesic action of tramadol is only partially inhibited by the opioid antagonist naloxone, which suggests the existence of an additional non-opioid mechanism of action. It has been found that monoaminergic activity, wherein noradrenaline and serotonin (5-HT) reuptake are inhibited, contributes significantly to the analgesic action of tramadol by blocking nociceptive impulses at the spinal level.

In a further embodiment, the administered compound is the (+)cis enantiomer of tramadol, set forth above.

In another embodiment, the substituted aminomethyl-phenyl-cyclohexane derivative is represented by the following structural Formula III in which the nitrogen of the aminomethyl group is in the form of the N-oxide:

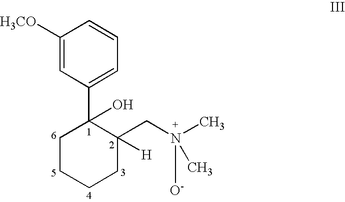

and enantiomers and mixtures thereof or pharmaceutically acceptable salts, solvates and hydrates thereof.

In a particular embodiment, the compound of Formula III is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula III is a 50:50 mixture of (+)cis and (−)cis enantiomers as shown below:

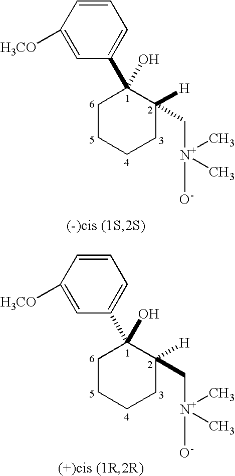

In other words, the compound of Formula III is the 50:50 mixture of the N-oxide of (+/−)cis-2-[(dimethylamino)methyl]-1-(3-methoxyphenyl) cyclohexanol.

In a further embodiment, the N-oxide is predominantly the (+)cis enantiomer, as set forth above.

In one embodiment, the substituted aminomethyl-phenyl-cyclohexane derivative suitable for use in the invention is represented by structural Formula IV:

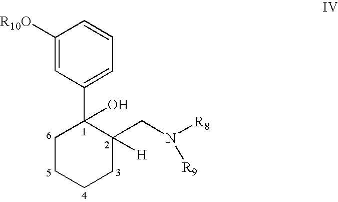

and enantiomers and mixtures thereof wherein:

R₈, R₉ and R₁₀ are independently hydrogen or an alkyl group;

or pharmaceutically acceptable salts, solvates or hydrates thereof.

In a particular embodiment, the compound of Formula IV is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula IV is a 50:50 mixture of (+)cis and (−)cis enantiomers as shown below:

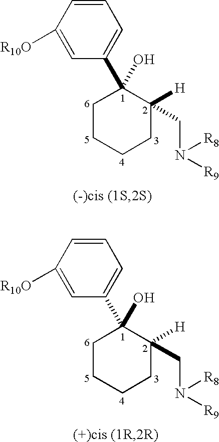

In a further embodiment, the compounds of Formula IV are predominantly the (+)cis enantiomer, as set forth above.

In a particular embodiment, R₁₀ is hydrogen. In a further embodiment wherein R₁₀ is hydrogen, R₈ and R₉ are independently hydrogen or an alkyl group, for example, a methyl group. When R₁₀ is hydrogen and R₈ and R₉ are methyl groups, and Formula IV is the racemic mixture of the (+)cis and (−)cis enantiomers, the compound can be referred to as O-desmethyltramadol. The specific (+) and (−) enantiomers set forth above, can be referred to as (+)O-desmethyltramadol and (−)O-desmethyltramadol.

In yet another embodiment, R₁₀ is hydrogen, R₈ is hydrogen and R₉ is a methyl group. When R₁₀ is hydrogen, R₈ is hydrogen and R₉ is a methyl group, and Formula IV is the racemic mixture of the (+)cis and (−)cis enantiomers, the compound can be referred to as O-desmethyl-N-mono-desmethyl-tramadol. The specific (+)cis and (−)cis enantiomers as set forth above can be referred to as (+)O-desmethyl-N-mono-desmethyl-tramadol and (−) O-desmethyl-N-mono-desmethyl-tramadol.

In another embodiment, the substituted aminomethyl-phenyl-cyclohexane derivative suitable for use in the invention is represented by structural Formula V:

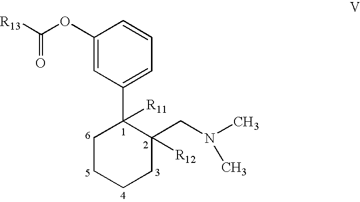

and enantiomers and mixtures thereof wherein:

R₁₁, is —OH;

R₁₂ is hydrogen or R₁₁ and R₁₂ together form a double bond;

R₁₃ is an aryl group selected from the group consisting of:

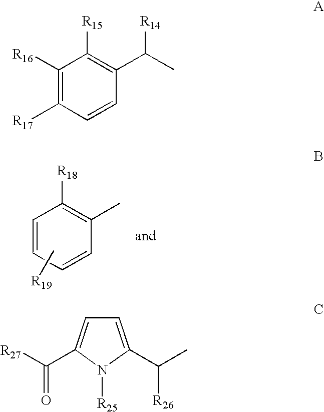

wherein:

R₁₄ is hydrogen or an alkyl group;

R₁₅ is hydrogen, —NH₂, —NHR₂₀ or —OR₂₀;

R₁₆ is hydrogen, —COR₂₀, —OR₂₀ or halogen;

R₁₇ is hydrogen, an alkyl group, —O-alkenyl, a phenyl group or R₁₆ and R₁₇ are —CH═CR₂, —CR₂₂═CH—, forming an aromatic ring;

R₁₈ is hydrogen, —COR₂₃, —OR₂₄ or a halogen;

R₁₉ is hydrogen, halogen, an alkyl group, —O-alkyl, —NO₂ or an aryl group;

R₂₀ is a phenyl group optionally substituted by one or more of the following: halogen, —NO₂, an alkyl group, an alkenyl group, —OH or —NH₂;

R₂₁, and R₂₂ are independently hydrogen or —O-alkyl;

R₂₃ is a phenyl group optionally substituted by one or more of the following: halogen, —NO₂, an alkyl group, an alkenyl group, —OH or —NH₂;

R₂₄ is hydrogen, —CO-alkyl (preferably methyl) or a phenyl group optionally substituted by one or more of the following: halogen, —NO₂, an alkyl group, an alkenyl group, —OH or —NH₂;

R₂₅ and R₂₆ are independently hydrogen, an alkyl group or form a —CH₂—CH₂— group;

R₂₇ is a phenyl group optionally substituted by one or more of the following: halogen, —NO₂, an alkyl group, an alkenyl group, —OH or —NH₂;

or pharmaceutically acceptable salts, solvates or hydrates thereof.

In a particular embodiment of Formula V, R₁₁ is —OH, R₁₂ is H and R₁₃ is:

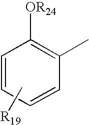

wherein:

R₂₄ is hydrogen or —COCH₃;

R₁₉ is halogen, an alkyl group, —O-alkyl or —NO₂.

It is preferred that when R₁₉ is —O-alkyl, the alkyl group is a methyl group.

It is preferred that when R₁₉ is an alkyl group, the alkyl group is substituted with one or more halogens. For example the substituted alkyl group is —CF₃.

Substituted aminomethyl-phenyl-cyclohexane derivatives in accordance with Formula V are further described in U.S. Pat. No. 6,455,585 B1 and published PCT Application WO 01/49650, which are incorporated herein by reference.

As used herein, lower urinary tract refers to all parts of the urinary tract except the kidneys.

As used herein, lower urinary tract disorder refers to any disorder involving the lower urinary tract, including but not limited to overactive bladder, interstitial cystitis, prostatitis, prostadynia and benign prostatic hyperplasia.

As used herein, bladder disorder refers to any condition involving the urinary bladder.

As used herein, overactive bladder refers to a chronic condition resulting from overactivity of the detrusor muscle, wherein the bladder initiates contraction too early while filling with urine, manifesting with one or more symptoms of urinary frequency, urinary urgency, urinary urge incontinence, nocturia or enuresis. Overactive bladder can be neurogenic or non-neurogenic.

Neurogenic overactive bladder (or neurogenic bladder) is a type of overactive bladder which occurs as a result of detrusor muscle overactivity referred to as detrusor hyperreflexia, secondary to neurologic disorders.

Non-neurogenic overactive bladder occurs as a result of detrusor muscle overactivity referred to as detrusor muscle instability. Detrusor muscle instability can arise from non-neurological abnormalities, such as bladder stones, muscle disease, urinary tract infection or drug side effects or can be idiopathic.

Interstitial cystitis is used herein in its conventional sense to refer to a disorder associated with symptoms that can include irritative voiding symptoms, urinary frequency, urgency, nocturia, suprapubic pain and/or pelvic pain related to and relieved by voiding.

As used herein, urinary frequency refers to urinating more frequently than the patient desires. As there is considerable interpersonal variation in the number of times in a day that an individual would normally expect to urinate, “more frequently than the patient desires” is further defined as a greater number of times per day than that patient's historical baseline. “Historical baseline” is further defined as the median number of times the patient urinated per day during a normal or desirable time period.

As used herein, urinary urgency refers to sudden strong urges to urinate with little or no chance to postpone the urination.

As used herein, incontinence refers to the inability to control excretory functions, including urination (urinary incontinence).

As used herein, urinary stress incontinence (also referred to as stress incontinence) refers to a medical condition in which urine leaks when a person coughs, sneezed, laughs, exercises, lifts heavy objects or does anything which puts pressure on the bladder.

As used herein, urinary urge incontinence (also referred to as urge incontinence) refers to the involuntary loss of urine associated with urinary urgency. It is understood that in some cases urge incontinence can be accompanied by stress incontinence, also referred to as mixed stress/urge incontinence. Thus, reference to the treatment of the symptom of urinary urge incontinence, can include treatment of urge incontinence in mixed stress/urge incontinence or urge incontinence.

As used herein, nocturia refers to being awakened from sleep to urinate more frequently than the patient desires.

As used herein, enuresis refers to involuntary voiding of urine which can be complete or incomplete. Nocturnal enuresis refers to enuresis which occurs during sleep. Diurnal enuresis refers to enuresis which occurs while awake.

As used herein, prostatitis refers to any type of disorder associated with inflammation of the prostate, including chronic and acute bacterial prostatitis and chronic non-bacterial prostatitis, and which is usually associated with symptoms of urinary frequency and/or urinary urgency.

Acute and chronic bacterial prostatitis are used herein in the conventional sense to refer to a disorder characterized by inflammation of the prostate and bacterial infection of the prostate gland, usually associated with symptoms of pain, urinary frequency and/or urinary urgency. Chronic bacterial prostatitis is distinguished from acute bacterial prostatitis based on the recurrent nature of the disorder. Chronic non-bacterial prostatitis is used herein in its conventional sense to refer to a disorder characterized by inflammation of the prostate which is of unknown etiology accompanied by the presence of an excessive amount of inflammatory cells in prostatic secretions not currently associated with bacterial infection of the prostate gland, and usually associated with symptoms of pain, urinary frequency and/or urinary urgency.

Prostadynia is a disorder which mimics the symptoms of prostatitis absent inflammation of the prostate, bacterial infection of the prostate and elevated levels inflammatory cells in prostatic secretions. Prostadynia can be associated with symptoms of pain, urinary frequency and/or urinary urgency.

Benign prostatic hyperplasia is used herein in its conventional sense to refer to a disorder associated with benign enlargement of the prostate gland which can be associated with urinary frequency, urinary urgency, urge incontinence, nocturia, and/or reduced urinary force and speed of flow.

In another embodiment, the method further comprises administering a therapeutically effective amount of an (i.e., one or more) additional therapeutic agent.

The invention relates to a method of treating at least one symptom of a lower urinary tract disorder in a subject in need of treatment wherein the symptom is selected from the group consisting of urinary frequency, urinary urgency, urinary urge incontinence, nocturia and enuresis comprising coadministering to said subject a first amount of an α₂δ subunit calcium channel ligand and a second amount of a substituted aminomethyl-phenyl-cyclohexane derivative, wherein the first and second amounts together comprise a therapeutically effective amount.

In one embodiment, coadministration of a first amount of an α₂δ subunit calcium channel ligand and a second amount of a substituted aminomethyl-phenyl-cyclohexane derivative can result in an enhanced or synergistic therapeutic effect, wherein the combined effect is greater than the additive effect resulting from separate administration of the first amount of the α₂δ subunit calcium channel ligand and the second amount of the substituted aminomethyl-phenyl-cyclohexane derivative.

In one embodiment, the lower urinary tract disorder can be selected from the group consisting of overactive bladder, interstitial cystitis, prostatitis, prostadynia and benign prostatic hyperplasia.

In another embodiment, the lower urinary tract disorder is overactive bladder.

In yet another embodiment, the lower urinary tract disorder is interstitial cystitis.

In another embodiment, the coadministration methods further comprise administering a therapeutically effective amount of an (i.e., one or more) additional therapeutic agent.

In one embodiment, the α₂δ subunit calcium channel ligand is a GABA analog. For example, the GABA analog can be selected from the group consisting of: gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and combinations thereof.

In a particular embodiment, the α₂δ subunit calcium channel ligand is gabapentin, pregabalin or a combination thereof.

In another embodiment, the substituted aminomethyl-phenyl-cyclohexane derivative is represented by structural Formula I:

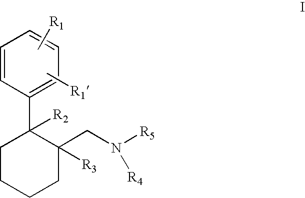

and enantiomers and mixtures thereof wherein:

R₁ and R₁′ are independently hydrogen, an aliphatic group, an aryl group, an arylalkyl group, a halogen, —CN, —OR, —SR₆, —NR₆R₆, —OC(O)R₆, —C(O)OR₆, —C(O)R₆ or —C(O)NR₆R₆;

R₂ is hydrogen, halogen, —OR₇ or —OC(O)R₇;

R₃ is hydrogen or an aliphatic group;

or R₂ and R₃ together form a double bond;

R₄ and R₅ are independently hydrogen, an aliphatic group, an aryl group or an arylalkyl group;

R₆ is hydrogen, an aliphatic group, an aryl group or an arylalkyl group;

R₇ is hydrogen, an aliphatic group, an aryl group or an arylalkyl group;

or pharmaceutically acceptable salts, solvates or hydrates thereof.

In a particular embodiment of Formula I, R₂ is —OH. When R₂ is —OH, it is preferred that R₁′ is hydrogen and R, is —OCH₃, preferably substituted at the meta position of the phenyl ring.

In a further embodiment of Formula I, R₂ is —OH, R₁′ is hydrogen and R₁ is —OR₆, substituted at the meta position of the phenyl ring and R₆ is an aliphatic group, for example, an alkyl group. In a particular embodiment, wherein R₂ is —OH, R₁′ is hydrogen and R₁ is —OR₆, substituted at the meta position of the phenyl ring and R₆ is an alkyl group, R₃, R₄ and R₅ can be hydrogen or an alkyl group.

In a particular embodiment, the α₂δ subunit calcium channel ligand is a GABA analog and the substituted aminomethyl-phenyl-cyclohexane is a compound of Formula I. In a specific embodiment, the GABA analog is selected from the group consisting of: gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and combinations thereof. It is preferred that the GABA analog is gabapentin, pregabalin or a combination thereof.

In yet another embodiment, the substituted aminomethyl-phenyl-cyclohexane derivative suitable for use in the invention is represented by structural Formula II:

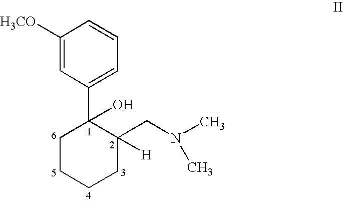

and enantiomers and mixtures thereof or pharmaceutically acceptable salts, solvates or hydrates thereof.

In a particular embodiment, the compound of Formula II is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula II is a 50:50 mixture of (+)cis and (−)cis enantiomers as shown below:

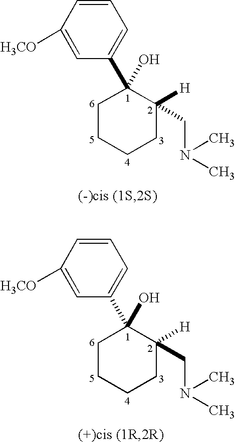

In other words, the compound of Formula II is the 50:50 mixture of (+/−)cis-2-[(dimethylamino)methyl]-1-(3-methoxyphenyl) cyclohexanol, commonly referred to as tramadol. The compound can be in the form of a pharmaceutically acceptable salt. Typically, tramadol is administered in the form of the hydrochloride salt. The tramadol hydrochloride is also known, for example, by the tradename ULTRAM®.

In a further embodiment, the administered compound is the (+)cis enantiomer of tramadol, set forth above.

In a particular embodiment, the α₂δ subunit calcium channel ligand is a GABA analog and the substituted aminomethyl-phenyl-cyclohexane is a compound of Formula II. In a specific embodiment, the GABA analog is selected from the group consisting of: gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and combinations thereof. It is preferred that the GABA analog is gabapentin, pregabalin or a combination thereof.

In still another embodiment, the substituted aminomethyl-phenyl-cyclohexane derivative is represented by the following structural Formula III in which the nitrogen of the aminomethyl group is in the N-oxide form:

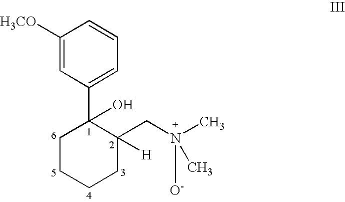

and enantiomers and mixtures thereof or pharmaceutically acceptable salts, solvates and hydrates thereof.

In a particular embodiment, the compound of Formula III is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula III is a 50:50 mixture of (+)cis and (−)cis enantiomers as shown below:

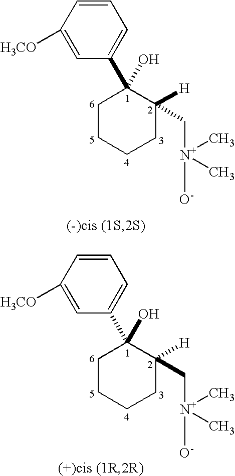

In other words, the compound of Formula III is the 50:50 mixture of the N-oxide of (+/−)cis-2-[(dimethylamino)methyl]-1-(3-methoxyphenyl) cyclohexanol.

In a further embodiment, the N-oxide is predominantly the (+)cis enantiomer, as set forth above.

In a particular embodiment, the α₂δ subunit calcium channel ligand is a GABA analog and the substituted aminomethyl-phenyl-cyclohexane is a compound of Formula III. In a specific embodiment, the GABA analog is selected from the group consisting of: gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and combinations thereof. It is preferred that the GABA analog is gabapentin, pregabalin or a combination thereof.

In yet a further embodiment, the substituted aminomethyl-phenyl-cyclohexane derivative suitable for use in the invention is represented by structural Formula IV:

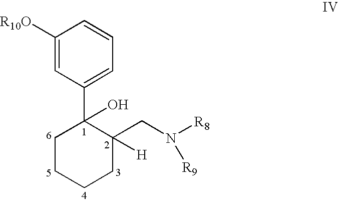

and enantiomers and mixtures thereof wherein:

R₈, R₉ and R₁₀ are independently hydrogen or an alkyl group;

or pharmaceutically acceptable salts, solvates or hydrates thereof.

In a particular embodiment, the compound of Formula IV is a mixture of the (+)cis and (−)cis enantiomers, wherein the C-1 and C-2 carbons of the cyclohexyl ring are (1R,2R) and (1S,2S), respectively, and the substituents on C-1 and C-2 are in the cis orientation.

In a specific embodiment, the mixture of the (+)cis and (−)cis enantiomers is a racemic mixture. That is, the compound of Formula IV is a 50:50 mixture of (+)cis and (−)cis enantiomers as shown below:

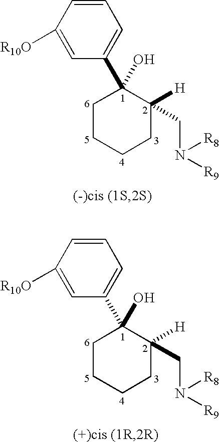

In a further embodiment, the compounds of Formula IV are predominantly the (+)cis enantiomer, as set forth above.

In a particular embodiment, R₁₀ is H. In a further embodiment wherein R₁₀ is hydrogen, R₈ and R₉ are independently hydrogen or an alkyl group, for example, a methyl group. When R₁₀ is hydrogen and R₈ and R₉ are methyl groups, and Formula IV is the racemic mixture of the (+)cis and (−)cis enantiomers, the compound can be referred to as O-desmethyltramadol. The specific (+)cis and (−)cis enantiomers set forth above, can be referred to as (+)O-desmethyl-tramadol and (−)O-desmethyltramadol.

In yet another embodiment, R₁₀ is hydrogen, R₈ is hydrogen and R₉ is a methyl group. When R₁₀ is hydrogen, R₈ is hydrogen and R₉ is a methyl group, and Formula IV is the racemic mixture of the (+)cis and (−)cis enantiomers, the compound can be referred to as O-desmethyl-N-mono-desmethyl-tramadol. The specific (+)cis and (−)cis enantiomers as set forth above can be referred to as (+)O-desmethyl-N-mono-desmethyl-tramadol and (−) O-desmethyl-N-mono-desmethyl-tramadol.

In a particular embodiment, the α₂δ subunit calcium channel ligand is a GABA analog and the substituted aminomethyl-phenyl-cyclohexane is a compound of Formula IV. In a specific embodiment, the GABA analog is selected from the group consisting of: gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and combinations thereof. It is preferred that the GABA analog is gabapentin, pregabalin or a combination thereof.

In another embodiment, the substituted aminomethyl-phenyl-cyclohexane derivative suitable for use in the invention is represented by structural Formula V:

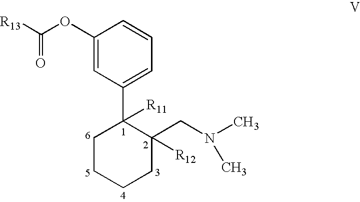

and enantiomers and mixtures thereof wherein:

R₁₁, is —OH;

R₁₂ is hydrogen or R₁₁ and R₁₂ together form a double bond;

R₁₃ is an aryl group selected from the group consisting of:

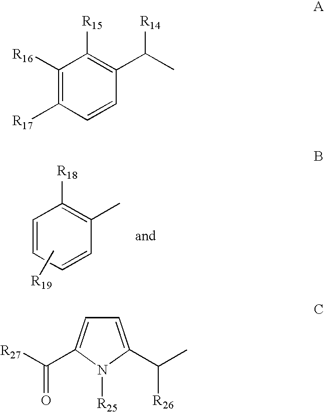

wherein:

R₁₄ is hydrogen or an alkyl group;

R₁₅ is hydrogen, —NH₂, —NHR₂₀ or —OR₂₀;

R₁₆ is hydrogen, —COR₂₀, —OR₂₀ or halogen;

R₁₇ is hydrogen, an alkyl group, —O-alkenyl, a phenyl group or R₁₆ and R₁₇ are —CH═CR₂, —CR₂₂═CH—, forming an aromatic ring;

R₁₈ is hydrogen, —COR₂₃, —OR₂₄ or a halogen;

R₁₉ is hydrogen, halogen, an alkyl group, —O-alkyl, —NO₂ or an aryl group;

R₂₀ is a phenyl group optionally substituted by one or more of the following: halogen, —NO₂, an alkyl group, an alkenyl group, —OH or —NH₂;

R₂₁ and R₂₂ are independently hydrogen or —O-alkyl;

R₂₃ is a phenyl group optionally substituted by one or more of the following: halogen, —NO₂, an alkyl group, an alkenyl group, —OH or —NH₂;

R₂₄ is hydrogen, —CO-alkyl (preferably methyl) or a phenyl group optionally substituted by one or more of the following: halogen, —NO₂, an alkyl group, an alkenyl group, —OH or —NH₂;

R₂₅ and R₂₆ are independently hydrogen, an alkyl group or form a —CH₂—CH₂— group;

R₂₇ is a phenyl group optionally substituted by one or more of the following: halogen, —NO₂, an alkyl group, an alkenyl group, —OH or —NH₂;

or pharmaceutically acceptable salts, solvates or hydrates thereof.

In a particular embodiment of Formula V, R₁₁ is —OH, R₁₂ is H and R₁₃ is:

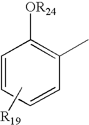

wherein:

R₂₄ is hydrogen or —COCH₃;

R₁₉ is halogen, an alkyl group, —O-alkyl or —NO₂.

It is preferred that when R₁₉ is —O-alkyl that the alkyl group is a methyl group.

It is preferred that when R₁₉ is an alkyl group, the alkyl group is substituted with one or more halogens. For example the substituted alkyl group is —CF₃.

In a particular embodiment, the α₂δ subunit calcium channel ligand is a GABA analog and the substituted aminomethyl-phenyl-cyclohexane is a compound of Formula V. In a specific embodiment, the GABA analog is selected from the group consisting of: gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and combinations thereof. It is preferred that the GABA analog is gabapentin, pregabalin or a combination thereof.

The invention further relates to pharmaceutical compositions useful for the treatment of at least one symptom of a lower urinary tract disorder in a subject in need of treatment wherein the symptom is selected from the group consisting of urinary frequency, urinary urgency, urinary urge incontinence, nocturia and enuresis. The pharmaceutical composition comprises a first amount of an α₂δ subunit calcium channel ligand and a second amount of a substituted aminomethyl-phenyl-cyclohexane derivative. Suitable α₂δ subunit calcium channel ligands and substituted aminomethyl-phenyl-cyclohexane derivatives include those described herein as suitable for use in the method. The pharmaceutical compositions of the present invention can optionally contain a pharmaceutically acceptable carrier. The first amount of an α₂δ subunit calcium channel ligand and the second amount of a substituted aminomethyl-phenyl-cyclohexane derivative can together comprise a therapeutically effective amount.

In one embodiment, the lower urinary tract disorder can be selected from the group consisting of overactive bladder, interstitial cystitis, prostatitis, prostadynia and benign prostatic hyperplasia.

In another embodiment, the lower urinary tract disorder is overactive bladder.

In yet another embodiment, the lower urinary tract disorder is interstitial cystitis.

In a further embodiment, the pharmaceutical composition further comprises an (i.e., one or more) additional therapeutic agent.

An additional therapeutic agent suitable for use in the methods and pharmaceutical compositions described herein, can be, but is not limited to, for example: an antimuscarinic (e.g., oxybutynin, DITROPAN®, tolterodine, flavoxate, propiverine, trospium); a muscosal surface protectant (e.g., ELMIRON®); an antihistamine (e.g., hydroxyzine hydrochloride or pamoate); an anticonvulsant (e.g., NEURONTIN® and KLONOPIN®); a muscle relaxant (e.g., VALIUM®); a bladder antispasmodic (e.g., URIMAX®); a tricyclic antidepressant (e.g., imipramine); a nitric oxide donor (e.g., nitroprusside), a β₃-adrenergic receptor agonist, a bradykinin receptor antagonist, a neurokinin receptor antagonist, a sodium channel modulator, such as TTX-R sodium channel modulator and/or activity dependent sodium channel modulator and a Cav2.2 subunit calcium channel modulator. Generally, the additional therapeutic agent will be one that is useful for treating the disorder of interest. Preferably, the additional therapeutic agent does not diminish the effects of the primary agent(s) and/or potentiates the effect of the primary agent(s).

Use of an additional therapeutic agent in combination with the primary agent(s) (i.e., α₂δ subunit calcium channel ligands and substituted aminomethyl-phenyl-cyclohexane derivatives) can result in less of any of the primary agent(s) and/or less of the additional agent being needed to achieve therapeutic efficacy. In some instances, use of less of an agent can be advantageous in that it provides a reduction in undesirable side effects.

By the term “antimuscarinic agent” as used herein is intended any muscarinic acetylcholine receptor antagonist. Unless otherwise indicated, the terms “anticholinergic agent,” “antinicotinic agent,” and “antimuscarinic agent” are intended to include anticholinergic, antinicotinic, and antimuscarinic agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

More specifically, oxybutynin, also known as 4-diethylaminio-2-butynyl phenylcyclohexyglycolate is a preferred antimuscarinic agent. It has the following structure:

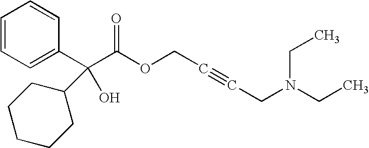

DITROPAN® (oxybutynin chloride) is the d,l racemic mixture of the above compound, which is known to exert antispasmodic effect on smooth muscle and inhibit the muscarinic action of acetylcholine on smooth muscle. Metabolites and isomers of oxybutynin have also been shown to have activity useful according to the present invention. Examples include, but are not limited to N-desethyl-oxybutynin and S-oxybutynin (see, e.g., U.S. Pat. Nos. 5,736,577 and 5,532,278).

Additional compounds that have been identified as antimuscarinic agents and are useful in the present invention include, but are not limited to:

a. Darifenacin (DARYON®) or acids, salts, enantiomers, analogs, esters, amides, prodrugs, active metabolites, and derivatives thereof;

b. Solifenacin or acids, salts, enantiomers, analogs, esters, amides, prodrugs, active metabolites, and derivatives thereof;

c. YM-905 (solifenacin succinate) or acids, salts, enantiomers, analogs, esters, amides, prodrugs, active metabolites, and derivatives thereof;

d. Solifenacin monohydrochloride or acids, salts, enantiomers, analogs, esters, amides, prodrugs, active metabolites, and derivatives thereof;

e. Tolterodine (DETROL®) or acids, salts, enantiomers, analogs, esters, amides, prodrugs, active metabolites, and derivatives thereof;

f Propiverine (DETRUNORM®) or acids, salts, enantiomers, analogs, esters, amides, prodrugs, active metabolites, and derivatives thereof;

g. Propantheline bromide (PRO-BANTHINE®) or acids, salts, enantiomers, analogs, esters, amides, prodrugs, active metabolites, and derivatives thereof;

h. Hyoscyamine sulfate (LEVSIN®, CYSTOSPAz®) or acids, salts, enantiomers, analogs, esters, amides, prodrugs, active metabolites, and derivatives thereof;

i. Dicyclomine hydrochloride (BENTYL®) or acids, salts, enantiomers, analogs, esters, amides, prodrugs, active metabolites, and derivatives thereof;

j. Flavoxate hydrochloride (URISPAS®) or acids, salts, enantiomers, analogs, esters, amides, prodrugs, active metabolites, and derivatives thereof;

k. d,l (racemic) 4-diethylamino-2-butynyl phenylcyclohexylglycolate or acids, salts, enantiomers, analogs, esters, amides, prodrugs, active metabolites, and derivatives thereof;

l. (R)-N,N-diisopropyl-3-(2-hydroxy-5-methylphenyl)-3-phenylpropanamine L-hydrogen tartrate or acids, salts, enantiomers, analogs, esters, amides, prodrugs, active metabolites, and derivatives thereof;

m. (+)-(1S,3′R)-quinuclidin-3′-yl-1-phenyl-1,2,3,4-tetrahydro-isoquinoline-2-carboxylate monosuccinate or acids, salts, enantiomers, analogs, esters, amides, prodrugs, active metabolites, and derivatives thereof;

n. alpha(+)-4-(Dimethylamino)-3-methyl-1,2-diphenyl-2-butanol proprionate or acids, salts, enantiomers, analogs, esters, amides, prodrugs, active metabolites, and derivatives thereof;

o 1-methyl-4-piperidyl diphenylpropoxyacetate or acids, salts, enantiomers, analogs, esters, amides, prodrugs, active metabolites, and derivatives thereof;

p. 3-hydroxyspiro[1H,5H-nortropane-8,1′-pyrrolidinium benzilate or acids, salts, enantiomers, analogs, esters, amides, prodrugs, active metabolites, and derivatives thereof;

q. 4 amino-piperidine containing compounds as disclosed in Diouf et al. (2002) Bioorg. Med. Chem. Lett. 12: 2535-9;

r. pirenzipine or acids, salts, enantiomers, analogs, esters, amides, prodrugs, active metabolites, and derivatives thereof;

s. methoctramine or acids, salts, enantiomers, analogs, esters, amides, prodrugs, active metabolites, and derivatives thereof;

t. 4-diphenylacetoxy-N-methyl piperidine methiodide;

u. tropicamide or acids, salts, enantiomers, analogs, esters, amides, prodrugs, active metabolites, and derivatives thereof;

v. (2R)-N-[1-(6-aminopyridin-2-ylmethyl)piperidin-4-yl]-2-[(1R)-3,3-difluorocyclopentyl]-2-hydroxy-2-phenylacetamide or acids, salts, enantiomers, analogs, esters, amides, prodrugs, active metabolites, and derivatives thereof;

w. PNU-200577 ((R)-N,N-diisopropyl-3-(2-hydroxy-5-hydroxymethylphenyl)-3-phenylpropanamine) or acids, salts, enantiomers, analogs, esters, amides, prodrugs, active metabolites, and derivatives thereof;

x. KRP-197 (4-(2-methylimidazolyl)-2,2-diphenylbutyramide) or acids, salts, enantiomers, analogs, esters, amides, prodrugs, active metabolites, and derivatives thereof;

y. Fesoterodine or acids, salts, enantiomers, analogs, esters, amides, prodrugs, active metabolites, and derivatives thereof; and

z. SPM 7605 (the active metabolite of Fesoterodine), or acids, salts, enantiomers, analogs, esters, amides, prodrugs, active metabolites, and derivatives thereof.

The identification of further compounds that have antimuscarinic activity and would therefore be useful in the present invention can be determined by performing muscarinic receptor binding specificity studies as described by Nilvebrant (2002) Pharmacol. Toxicol. 90: 260-7 or cystometry studies as described by Modiri et al. (2002) Urology 59: 963-8.

The term “β₃ adrenergic receptor agonist” is used in its conventional sense to refer to a compound that binds to and agonizes β₃ adrenergic receptors. Unless otherwise indicated, the term “β₃ adrenergic receptor agonist” is intended to include β₃ adrenergic agonist agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Compounds that have been identified as β₃ adrenergic agonist agents and are useful in the present invention include, but are not limited to:

a. TT-138 and phenylethanolamine compounds as disclosed in U.S. Pat. No. 6,069,176, PCT Publication No. WO 97/15549 and available from Mitsubishi Pharma Corp., or acids, salts, esters, amides, prodrugs, active metabolites, and other derivatives thereof;

b. FR-149174 and propanolamine derivatives as disclosed in U.S. Pat. Nos. 6,495,546 and 6,391,915 and available from Fujisawa Pharmaceutical Co., or acids, salts, esters, amides, prodrugs, active metabolites, and other derivatives thereof;

c. KUC-7483, available from Kissei Pharmaceutical Co., or acids, salts, esters, amides, prodrugs, active metabolites, and other derivatives thereof,

d. 4′-hydroxynorephedrine derivatives such as 2-2-chloro-4-(2-((1S,2R)-2-hydroxy-2-(4-hydroxyphenyl)-1-methylethylamino)ethyl)-phenoxy acetic acid as disclosed in Tanaka et al. (2003) J. Med. Chem. 46: 105-12 or acids, salts, esters, amides, prodrugs, active metabolites, and other derivatives thereof;

e. 2-amino-1-phenylethanol compounds, such as BRL35135 ((R*R*)-(.+−.)-[4-[2-[2-(3-chlorophenyl)-2-ydroxyethylamino]propyl]phenoxy]acetic acid methyl ester hydrobromide salt as disclosed in Japanese Patent Publication No. 26744 of 1988 and European Patent Publication No. 23385), and SR58611A ((RS)-N-(7-ethoxycarbonylmethoxy-1,2,3,4-tetrahydronaphth-2-yl)-2-(3-chlorophenyl)-2-hydroxyethanamine hydrochloride as disclosed in Japanese Laid-open Patent Publication No. 66152 of 1989 and European Laid-open Patent Publication No. 255415) or acids, salts, esters, amides, prodrugs, active metabolites, and other derivatives thereof;

f. GS 332 (Sodium (2R)-[3-[3-[2-(3Chlorophenyl)-2-hydroxyethylamino]cyclohexyl]phenoxy]acetate) as disclosed in Iizuka et al. (1998) J. Smooth Muscle Res. 34: 139-49 or acids, salts, esters, amides, prodrugs, active metabolites, and other derivatives thereof;

g. BRL-37,344 (4-[-[(2-hydroxy-(3-chlorophenyl)ethyl)-amino]propyl]phenoxyacetate) as disclosed in Tsujii et al. (1998) Physiol. Behav. 63: 723-8 and available from GlaxoSmithKline or acids, salts, esters, amides, prodrugs, active metabolites, and other derivatives thereof;

h. BRL-26830A as disclosed in Takahashi et al. (1992) Jpn Circ. J. 56: 936-42 and available from GlaxoSmithKline or acids, salts, esters, amides, prodrugs, active metabolites, and other derivatives thereof;

i. CGP 12177 (4-[3-t-butylamino-2-hydroxypropoxy]benzimidazol-2-one) (a ½ adrenergic antagonist reported to act as an agonist for the 3 adrenergic receptor) as described in Tavernier et al. (1992) J. Pharmacol. Exp. Ther. 263: 1083-90 and available from Ciba-Geigy or acids, salts, esters, amides, prodrugs, active metabolites, and other derivatives thereof;

j. CL 316243 (R,R-5-[2-[[2-(3-chlorophenyl)-2-hydroxyethyl]amino]propyl]-1,3- benzodioxole-2,2-dicarboxylate) as disclosed in Berlan et al. (1994) J. Pharmacol. Exp. Ther. 268: 1444-51 or acids, salts, esters, amides, prodrugs, active metabolites, and other derivatives thereof;

k. Compounds having 3 adrenergic agonist activity as disclosed in U.S. Patent Application 20030018061 or acids, salts, esters, amides, prodrugs, active metabolites, and other derivatives thereof;

l. ICI 215,001 HCl ((S)-4-[2-Hydroxy-3-phenoxypropyl-aminoethoxy]phenoxyacetic acid hydrochloride) as disclosed in Howe (1993) Drugs Future 18: 529 and available from AstraZeneca/ICI Labs or acids, salts, enantiomers, analogs, esters, amides, prodrugs, active metabolites, and derivatives thereof;

m. ZD 7114 HCl (ICI D7114; (S)-4-[2-Hydroxy-3-phenoxypropyl-aminoethoxy]-N-(2-methoxyethyl)phenoxyacetamide HCl) as disclosed in Howe (1993) Drugs Future 18: 529 and available from AstraZeneca/ICI Labs or acids, salts, enantiomers, analogs, esters, amides, prodrugs, active metabolites, and derivatives thereof;

n. Pindolol (1-(1H-Indol-4-yloxy)-3-[(1-methylethyl)amino]-2-propanol) as disclosed in Blin et al (1994) Mol.Pharmacol. 44: 1094 or acids, salts, enantiomers, analogs, esters, amides, prodrugs, active metabolites, and derivatives thereof;

o. (S)-(−)-Pindolol ((S)-1-(1H-indol-4-yloxy)-3-[(1-methylethyl)amino]-2-propanol) as disclosed in Walter et al (1984) Naunyn-Schmied.Arch.Pharmacol. 327: 159 and Kalkman (1989) Eur.J.Pharmacol. 173: 121 or acids, salts, enantiomers, analogs, esters, amides, prodrugs, active metabolites, and derivatives thereof;

p. SR 59230A HCl (1-(2-Ethylphenoxy)-3-[[(1S)-1,2,3,4-tetrahydro-1-naphthalenyl]amino]-(2S)-2-propanol hydrochloride) as disclosed in Manara et al. (1995) Pharmacol. Comm. 6: 253 and Manara et al. (1996) Br. J. Pharmacol. 117: 435 and available from Sanofi-Midy or acids, salts, enantiomers, analogs, esters, amides, prodrugs, active metabolites, and derivatives thereof;

q. SR 58611 (N[2s)7-carb-ethoxymethoxy-1,2,3,4-tetra-hydronaphth]-(2r)-2-hydroxy-2(3-chlorophenyl) ethamine hydrochloride) as disclosed in Gauthier et al. (1999) J. Pharmacol. Exp. Ther. 290: 687-693 and available from Sanofi Research; and

r. YM178 available from Yamanouchi Pharmaceutical Co. or acids, salts, esters, amides, prodrugs, active metabolites, and other derivatives thereof.

s. N-[4-[2-[2(R)-Hydroxy-2-(3-pyridyl)ethylamino]ethyl]phenyl]-4-[4-[4-(trifluoromethyl)phenyl]thiazo benzenesulfonamide dihydrochloride having CAS Registry No. 211031-81-1 (free base Registry No. 211031-01-5), referred to as L-796568.

The identification of further compounds that have β₃ adrenergic agonist activity and would therefore be useful in the present invention can be determined by performing radioligand binding assays and/or contractility studies as described by Zilberfarb et al. (1997) J. Cell Sci. 110: 801-807; Takeda et al. (1999) J. Pharmacol. Exp. Ther. 288: 1367-1373; and Gauthier et al. (1999) J. Pharmacol. Exp. Ther. 290: 687-693.

Further, agents for use as additional therapeutic agents include sodium channel modulators, such as TTX-R sodium channel modulators and/or activity dependent sodium channel modulators. TTX-R sodium channel modulators for use in the present invention include but are not limited to compounds that modulate or interact with Nav1.8 and/or Nav1.9 channels.

Sodium channel modulators suitable for use as in the practice of the invention include, but are not limited to propionamides such as Ralfinamide (NW-1029) (as disclosed in U.S. Pat. Nos. 5,236,957 and 5,391,577), which is also known as (+)-2(S)-[4-(2-Fluorobenzyloxy)benzylamino]propionamide and safinamide (as disclosed in U.S. Pat. Nos. 5,236,957 and 5,391,577), which is also known as 2(S)-[4-(3-Fluorobenzyloxy)benzylamino]propionamide methanesulfonate

Further sodium channel modulators include for example, N-phenylalkyl substituted a-amino carboxamide derivatives in addition to Ralfinamide and Salfinamide as disclosed in U.S. Pat. No. 5,236,957; Other N-phenylalkyl substituted α-amino carboxamide derivatives in addition to Ralfinamide and Salfinamide as disclosed in U.S. Pat. No. 5,391,577; Substituted 2-benzylamino-2-phenyl-acetamide compounds as disclosed in U.S. Pat. No. 6,303,819; aryldiazines and aryltriazines such as: sipatrigine (BW-619C; as disclosed in U.S. Pat. No. 5,684,005), which is also known as 4-Amino-2-(4-methylpiperazin-1-yl)-5-(2,3,5-trichlorophenyl)pyrimidine; 2-(4-Methylpiperazin-1-yl)-5-(2,3,5-trichlorophenyl)pyrimidine-4-amine; lamotrigine (as disclosed in U.S. Pat. No. 4,602,017), which is also known as 6-(2,3-Dichlorophenyl)-1,2,4-triazine-3,5-diamine; GW-273293 (as disclosed in U.S. Pat. No. 6,599,905), which is also known as 3-(2,3,5-Trichlorophenyl)pyrazine-2,6-diamine; 4030W92 (as disclosed in U.S. Pat. No. 6,124,308), which is also known as 5-(2,3-Dichlorophenyl)-6-(fluoromethyl)pyrimidine-2,4-diamine; Carbamazepine (as disclosed in U.S. Pat. No. 2,948,718), which is also known as 5H-Dibenz[d,f]azepine-5-carboxamide; Oxcarbazepine (as disclosed in U.S. Pat. No. 3,642,775), which is also known as 10-Oxo-10,11-dihydro-5H-dibenz[b,f]azepine-5-carboxamide; licarbazepine (as disclosed in DE 2011045), which is also known as (±)-10-Hydroxy-10,1 1-dihydro-5H-dibenz[b,f]azepine-5-carboxamide; BIA-2-093 (as disclosed in U.S. Pat. No. 5,753,646), which is also known as Acetic acid 5-carbamoyl-10,11-dihydro-5H-dibenzo[b,f]azepin-10(S)-yl ester; ADCI (as disclosed in U.S. Pat. No. 5,196,415), which is also known as (±)-5,10-Imino-10,11-dihydro-5H-dibenzo[a,d]cycloheptene-5-carboxamide; Phenytoin sodium (as disclosed in U.S. Pat. No. 2,409,754) and OROS®-Phenytoin (as disclosed in U.S. Pat. No. 4,260,769), which are also known as 5,5-Diphenylhydantoin sodium salt and 5,5-Diphenyl-2,4-imidazolidinedione salt; Fosphenytoin sodium (as disclosed in U.S. Pat. No. 4,260,769) and phosphenytoin sodium, which are also known as 3-(Hydroxymethyl)-5,5-diphenylhydantoin phosphate ester disodium salt and 5,5-Diphenyl-3-[(phosphonooxy)methyl]-2,4-imidazolidinedione disodium salt; Pilsicainide hydrochloride and analogs thereof (as disclosed in U.S. Pat. No. 4,564,624), which is also known as N-(2,6-Dimethylphenyl)-8-pyrrolizidineacetamide hydrochloride; N-(2,6-Dimethylphenyl)-1-azabicyclo[3.3.0]octane-5-acetamide hydrochloride; Tocainide (as disclosed in DE 2235745), which is also known as 2-Amino-N-(2,6-dimethylphenyl)propanamide hydrochloride; Flecainide (as disclosed in U.S. Pat. No. 3,900,481), which is also known as N-(2-Piperidylmethyl)-2,5-bis(2,2,2-trifluoroethoxy)benzamide monoacetate; mexiletine hydrochloride (as disclosed in U.S. Pat. No. 3,954,872), which is also known as 1-(2,6-Dimethylphenoxy)-2-propanamine hydrochloride; Ropivacaine hydrochloride (as disclosed in PCT Publication No. WO 85/00599), which is also known as (−)-(S)-N-(n-Propyl)piperidine-2-carboxylic acid 2,6-xylidide hydrochloride monohydrate; (−)-(S)-N-(2,6-Dimethylphenyl)-1-propylpiperidine-2-carboxamide hydrochloride monohydrate; (−)-(S)-1-Propyl-2′,6′-pipecoloxylidide hydrochloride monohydrate; Lidocaine (as disclosed in U.S. Pat. No. 2,441,498), which is also known as 2-(diethylamino)-N-(2,6-dimethylphenyl)acetamide; mepivacaine (as disclosed in U.S. Pat. No. 27,996,79), which is also known as N-(2,6-dimethylphenyl)-1-methyl-2-piperidinecarboxamide; bupivacaine (as disclosed in U.S. Pat. No. 2,955,111), which is also known as 1-butyl-N-(2,6-dimethylphenyl)-2-piperidinecarboxamide; Prilocaine (as disclosed in U.S. Pat. No. 3,160,662), also known as N-(2-methylphenyl)-2-(propylamino)propanamide; etidocaine (as disclosed in U.S. Pat. No. 3,812,147), which is also known as N-(2,6-dimethylphenyl)-1-methyl-2-piperidinecarboxamide; tetracaine (as disclosed in U.S. Pat. No. 1,889,645), which is also known as 4-(butylamino)benzoic acid 2-(diethylamino)ethyl ester; dibucaine (as disclosed in U.S. Pat. No. 1,825,623), which is also known as 2-butoxy-N-[2-(diethylamino)-ethyl]-4-quinolinecarboxamide; Soretolide, which is also known as 2,6-Dimethyl-N-(5-methylisozaxol-3-yl)benzamide; RS-132943 (as disclosed in U.S. Pat. No. 6,110,937), which is also known as 3(S)-(4-Bromo-2,6-dimethylphenoxymethyl)-1-methylpiperidine hydrochloride

The identification of other agents that have affinity for TTX-R sodium channels or proteins associated with TTX-R sodium channels and would be useful in the present invention can be determined by methods that measure functional TTX-R channel activity such as sodium flux as disclosed in Stallcup, W B (1979) J. Physiol. 286: 525-40 or electrophysiological approaches as disclosed in Weiser and Wilson (2002) Mol. Pharmacol. 62: 433-438. The identification of other agents that exhibit activity-dependent modulation of sodium channels and would be useful in the present invention can be determined by methods as disclosed in Li et al., (1999) Molecular Pharmacology 55:134-141.

Further, agents for use as additional therapeutic agents include “Cav2.2 subunit calcium channel modulators” which are capable of binding to the Cav2.2 subunit of a calcium channel to produce a physiological effect, such as opening, closing, blocking, up-regulating expression, or down-regulating expression of the channel. Unless otherwise indicated, the term “Cav2.2 subunit calcium channel modulator” is intended to include amino acid compounds, peptide, nonpeptide, peptidomimetic, small molecular weight organic compounds, and other compounds that modulate or interact with the Cav2.2 subunit of a calcium channel (e.g., a binding event) or proteins associated with the Cav2.2 subunit of a calcium channel (e.g., a binding event) such as anchor proteins, as well as salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Cav2.2 subunit calcium channel modulator useful as an additional therapeutic agent in the practice of the invention include, but are not limited to:

a. ω-conotoxin GVIA or a salt, enantiomer, analog, ester, amide, prodrug, active metabolite, or derivative thereof;

b. ω-conotoxin MVIIA or a salt, enantiomer, analog, ester, amide, prodrug, active metabolite, or derivative thereof;

c. ω-conotoxin CNVIIA or a salt, enantiomer, analog, ester, amide, prodrug, active metabolite, or derivative thereof;

d. ω-conotoxin CVIID or a salt, enantiomer, analog, ester, amide, prodrug, active metabolite, or derivative thereof;

e. ω-conotoxin AM336 or a salt, enantiomer, analog, ester, amide, prodrug, active metabolite, or derivative thereof;

f. Cilnidipine or a salt, enantiomer, analog, ester, amide, prodrug, active metabolite, or derivative thereof;

g. Amlodipine or a salt, enantiomer, analog, ester, amide, prodrug, active metabolite, or derivative thereof;

h. L-cysteine derivative 2A or a salt, enantiomer, analog, ester, amide, prodrug, active metabolite, or derivative thereof;

i.ω-agatoxin IVA or a salt, enantiomer, analog, ester, amide, prodrug, active metabolite, or derivative thereof;

j. N,N-dialkyl-dipeptidylamines or a salt, enantiomer, analog, ester, amide, prodrug, active metabolite, or derivative thereof;

k. Levetiracetam or a salt, enantiomer, analog, ester, amide, prodrug, active metabolite, or derivative thereof; and

l. Ziconotide (SNX-111) or a salt, enantiomer, analog, ester, amide, prodrug, active metabolite, or derivative thereof;

m. (S)-alpha-ethyl-2-oxo-1-pyrrolidineacetamide (illustrated below) and disclosed in U.S. Pat. Nos. 4,943,639, 4,837,223, and 4,696,943, or a salt, enantiomer, analog, ester, amide, prodrug, active metabolite, or derivative, thereof;

n. Substituted peptidylamines as disclosed in PCT Publication No. WO 98/54123, or a salt, enantiomer, analog, ester, amide, prodrug, active metabolite, or derivative, thereof;

o. PD-173212 or a salt, enantiomer, analog, ester, amide, prodrug, active metabolite, or derivative, thereof;

p. Reduced dipeptide analogues as disclosed in U.S. Pat. No. 6,316,440 and PCT Publication No. WO 00/06559, or a salt, enantiomer, analog, ester, amide, prodrug, active metabolite, or derivative, thereof;

q. Amino acid derivatives as disclosed in PCT Publication No. WO 99/02146, or a salt, enantiomer, analog, ester, amide, prodrug, active metabolite, or derivative, thereof;

r. Benzazepine derivatives as disclosed in Japanese Publication No. JP 2002363163, or a salt, enantiomer, analog, ester, amide, prodrug, active metabolite, or derivative, thereof;

s. Compounds disclosed in PCT Publication No. WO 02/36567, or a salt, enantiomer, analog, ester, amide, prodrug, active metabolite, or derivative, thereof;

t. Compounds disclosed in PCT Publication No. WO 03/018561, or a salt, enantiomer, analog, ester, amide, prodrug, active metabolite, or derivative, thereof;

u. Compounds disclosed in U.S. Patent Publication No. 2004009991 and PCT Publication No. WO 02/22588, or a salt, enantiomer, analog, ester, amide, prodrug, active metabolite, or derivative, thereof;

v. Dihydropyridine derivatives as disclosed in U.S. Pat. No. 6,610,717, U.S. Patent Publication No. 2002193605, and PCT Publication No. WO 00/78720, or a salt, enantiomer, analog, ester, amide, prodrug, active metabolite, or derivative, thereof;

w. Diarylalkene and diarylalkane derivatives as disclosed in PCT Publication No. WO 03/018538, or a salt, enantiomer, analog, ester, amide, prodrug, active metabolite, or derivative, thereof.

Cav2.2 subunit calcium channel modulator useful as an additional therapeutic agent in the practice of the invention include, but are not limited to non-peptide, and peptidomimetic drug-like molecules that bind to Cav2.2-containing calcium channels as disclosed in Lewis et al. (2000) J. Biol. Chem. 10: 35335-44; Smith et al. (2002) Pain 96: 119-27; Takahara et al. (2002) Eur. J. Pharmacol. 434: 43-7; Favreau et al. (2001) Biochemistry, 40: 14567-575; Seko et al. (2001) Bioorg. Med. Chem. Lett. 11: 2067-70; Hu et al. (2000) Bioorg. Med. Chem. Lett. 8: 1203-12; Lew et al. (1997) J. Biol. Chem. 272: 12014-23. It is understood that the present invention also encompasses any pharmaceutically acceptable, pharmacologically active salts, enantiomers, analogs, esters, amides, prodrugs, active metabolites, and derivatives of the aforementioned compounds.

The identification of other agents that have affinity for the Cav2.2 subunit of a calcium channel and would be useful in the present invention can be determined by performing Cav2.2 subunit binding affinity, electrophysiolgic, and/or other screening methods as described in Feng et al. (J. Biol. Chem., 278: 20171-20178, 2003), Feng et al. (J. Biol. Chem., 276: 15728-15735, 2001), Favreau et al. (Biochemistry, 40: 14567-575, 2001), and/or U.S. Pat. No. 6,387,897 assigned to NeuroMed Technologies Inc.

The term “spasmolytic” (also known as “antispasmodic”) is used in its conventional sense to refer to a compound that relieves or prevents muscle spasms, especially of smooth muscle. Unless otherwise indicated, the term “spasmolytic” is intended to include spasmolytic agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active. In general, spasmolytics have been implicated as having efficacy in the treatment of bladder disorders (See. e.g., Takeda et al. (2000) J. Pharmacol. Exp. Ther. 293: 939-45).

Compounds that have been identified as spasmolytic agents and are useful in the present invention include, but are not limited to:

a. α-α-diphenylacetic acid-4-(N-methyl-piperidyl) esters as disclosed in U.S. Pat. No. 5,897,875 or acids, salts, enantiomers, analogs, esters, amides, prodrugs, active metabolites, and derivatives thereof;

b. Human and porcine spasmolytic polypeptides in glycosylated form and variants thereof as disclosed in U.S. Pat. No. 5,783,416 or acids, salts, enantiomers, analogs, esters, amides, prodrugs, active metabolites, and derivatives thereof;

c. Dioxazocine derivatives as disclosed in U.S. Pat. No. 4,965,259 or acids, salts, enantiomers, analogs, esters, amides, prodrugs, active metabolites, and derivatives thereof;

d. Quaternary 6,11-dihydro-dibenzo-[b,e]-thiepine-1-N-alkylnorscopine ethers as disclosed in U.S. Pat. No. 4,608,377 or acids, salts, enantiomers, analogs, esters, amides, prodrugs, active metabolites, and derivatives thereof;

e. Quaternary salts of dibenzo[1,4]diazepinones, pyrido-[1,4]benzodiazepinones, pyrido[1,5]benzodiazepinones as disclosed in U.S. Pat. No. 4,594,190 or acids, salts, enantiomers, analogs, esters, amides, prodrugs, active metabolites, and derivatives thereof;

f. Endo-8,8-dialkyl-8-azoniabicyclo (3.2.1) octane-6,7-exo-epoxy-3-alkyl-carboxylate salts as disclosed in U.S. Pat. No. 4,558,054 or acids, salts, enantiomers, analogs, esters, amides, prodrugs, active metabolites, and derivatives thereof;

g. Pancreatic spasmolytic polypeptides as disclosed in U.S. Pat. No. 4,370,317 or acids, salts, enantiomers, analogs, esters, amides, prodrugs, active metabolites, and derivatives thereof;

h. Triazinones as disclosed in U.S. Pat. No. 4,203,983 or acids, salts, enantiomers, analogs, esters, amides, prodrugs, active metabolites, and derivatives thereof;

i. 2-(4-Biphenylyl)-N-(2-diethylamino alkyl)propionamide as disclosed in U.S. Pat. No. 4,185,124 or acids, salts, enantiomers, analogs, esters, amides, prodrugs, active metabolites, and derivatives thereof;

j. Piperazino-pyrimidines as disclosed in U.S. Pat. No. 4,166,852 or acids, salts, enantiomers, analogs, esters, amides, prodrugs, active metabolites, and derivatives thereof;

k. Aralkylamino carboxylic acids as disclosed in U.S. Pat. No. 4,163,060 or acids, salts, enantiomers, analogs, esters, amides, prodrugs, active metabolites, and derivatives thereof;

l. Aralkylamino sulfones as disclosed in U.S. Pat. No. 4,034,103 or acids, salts, enantiomers, analogs, esters, amides, prodrugs, active metabolites, and derivatives thereof;

m. Smooth muscle spasmolytic agents as disclosed in U.S. Pat. No. 6,207,852 or acids, salts, enantiomers, analogs, esters, amides, prodrugs, active metabolites, and derivatives thereof; and

n. Papaverine or acids, salts, enantiomers, analogs, esters, amides, prodrugs, active metabolites, and derivatives thereof.

The identification of further compounds that have spasmolytic activity and would therefore be useful in the present invention can be determined by performing bladder strip contractility studies as described in U.S. Pat. No. 6,207,852; Noronha-Blob et al. (1991) J. Pharmacol. Exp. Ther.256: 562-567; and/or Kachur et al. (1988) J. Pharmacol. Exp. Ther.247: 867-872.

The term “neurokinin receptor antagonist” is used in its conventional sense to refer to a compound that binds to and antagonizes neurokinin receptors. Unless otherwise indicated, the term “neurokinin receptor antagonist” is intended to include neurokinin receptor antagonist agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Suitable neurokinin receptor antagonists for use in the present invention that act on the NK1 receptor include, but are not limited to:

1-imino-2-(2-methoxy-phenyl)-ethyl)-7,7-diphenyl-4-perhydroisoindolone(3aR,7aR) (“RP 67580”); 2S,3S-cis-3-(2-methoxybenzylamino)-2-benzhydrylquinuclidine (“CP 96,345”); and (aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione)(“TAK-637”). Suitable neurokinin receptor antagonists for use in the present invention that act on the NK2 receptor include but are not limited to: ((S)-N-methyl-N-4-(4-acetylamino-4-phenylpiperidino)-2-(3,4-dichloropheny l)butylbenzamide (“SR 48968”); Met-Asp-Trp-Phe-Dap-Leu (“MEN 10,627”); and cyc(Gln-Trp-Phe-Gly-Leu-Met) (“L 659,877”). Suitable neurokinin receptor antagonists for use in the present invention also include acids, salts, esters, amides, prodrugs, active metabolites, and other derivatives of any of the agents mentioned above. The identification of further compounds that have neurokinin receptor antagonist activity and would therefore be useful in the present invention can be determined by performing binding assay studies as described in Hopkins et al. (1991) Biochem. Biophys. Res. Comm. 180: 1110-1117; and Aharony et al. (1994) Mol. Pharmacol. 45: 9-19.

The term “bradykinin receptor antagonist” is used in its conventional sense to refer to a compound that binds to and antagonizes bradykinin receptors. Unless otherwise indicated, the term “bradykinin receptor antagonist” is intended to include bradykinin receptor antagonist agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active. Suitable bradykinin receptor antagonists for use in the present invention that act on the B1 receptor include but are not limited to: des-arg10HOE 140 (available from Hoechst Pharmaceuticals) and des-Arg9bradykinin (DABK). Suitable bradykinin receptor antagonists for use in the present invention that act on the B2 receptor include but are not limited to: D-Phe⁷-BK; D-Arg-(Hyp³-Thi^{5,8}-D-Phe⁷)-BK (“NPC 349”); D-Arg-(Hyp³-D-Phe⁷)-BK (“NPC 567”); D-Arg-(Hyp³-Thi⁵-D-Tic⁷-Oic⁸)-BK (“HOE 140”); H-DArg-Arg-Pro-Hyp-Gly-Thi-c(Dab-DTic-Oic-Arg)c(7gamma-10alpha) (“MEN11270”); H-DArg-Arg-Pro-Hyp-Gly-Thi-Ser-DTic-Oic-Arg-OH(“Icatibant”); (E)-3-(6-acetamido-3-pyridyl)-N-[N-[2, 4-dichloro-3-[(2-methyl-8-quinolinyl)oxymethyl]phenyl]-N-methylaminocarbonylmethyl]acrylamide (“FR173567”); and WIN 64338. These compounds are more fully described in Perkins, M. N., et. al., Pain, supra; Dray, A., et. al., Trends Neurosci., supra; and Meini et al. (2000) Eur. J. Pharmacol. 388: 177-82. Suitable bradykinin receptor antagonists for use in the present invention also include acids, salts, esters, amides, prodrugs, active metabolites, and other derivatives of any of the agents mentioned above. The identification of further compounds that have bradykinin receptor antagonist activity and would therefore be useful in the present invention can be determined by performing binding assay studies as described in Manning et al. (1986) J. Pharmacol. Exp. Ther. 237: 504 and U.S. Pat. No. 5,686,565.

The term “nitric oxide donor” is used in its conventional sense to refer to a compound that releases free nitric oxide when administered to a patient. Unless otherwise indicated, the term “nitric oxide donor” is intended to include nitric oxide donor agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Suitable nitric oxide donors for the practice of the present invention include but are not limited to:

a. Nitroglycerin or acids, salts, enantiomers, analogs, esters, amides, prodrugs, active metabolites, and derivatives thereof;

b. Sodium nitroprusside or acids, salts, enantiomers, analogs, esters, amides, prodrugs, active metabolites, and derivatives thereof;

c. FK 409 (NOR-3) or acids, salts, enantiomers, analogs, esters, amides, prodrugs, active metabolites, and derivatives thereof;

d. FR 144420 (NOR-4) or acids, salts, enantiomers, analogs, esters, amides, prodrugs, active metabolites, and derivatives thereof,

e. 3-morpholinosydnonimine or acids, salts, enantiomers, analogs, esters, amides, prodrugs; active metabolites, and derivatives thereof;

f. Linsidomine chlorohydrate (“SIN-1”) or acids, salts, enantiomers, analogs, esters, amides, prodrugs, active metabolites, and derivatives thereof;

g. S-nitroso-N-acetylpenicillamine (“SNAP”) or acids, salts, enantiomers, analogs, esters, amides, prodrugs, active metabolites, and derivatives thereof;

h. AZD3582 (CINOD lead compound, available from NicOx S.A.) or acids, salts, enantiomers, analogs, esters, amides, prodrugs, active metabolites, and derivatives thereof;

i. NCX 4016 (available from NicOx S.A.) or acids, salts, enantiomers, analogs, esters, amides, prodrugs, active metabolites, and derivatives thereof;

j. NCX 701 (available from NicOx S.A.) or acids, salts, enantiomers, analogs, esters, amides, prodrugs, active metabolites, and derivatives thereof;

k. NCX 1022 (available from NicOx S.A.) or acids, salts, enantiomers, analogs, esters, amides, prodrugs, active metabolites, and derivatives thereof;

l. HCT 1026 (available from NicOx S.A.) or acids, salts, enantiomers, analogs, esters, amides, prodrugs, active metabolites, and derivatives thereof;

m. NCX 1015 (available from NicOx S.A.) or acids, salts, enantiomers, analogs, esters, amides, prodrugs, active metabolites, and derivatives thereof;

n. NCX 950 (available from NicOx S.A.) or acids, salts, enantiomers, analogs, esters, amides, prodrugs, active metabolites, and derivatives thereof;

o. NCX 1000 (available from NicOx S.A.) or acids, salts, enantiomers, analogs, esters, amides, prodrugs, active metabolites, and derivatives thereof;

p. NCX 1020 (available from NicOx S.A.) or acids, salts, enantiomers, analogs, esters, amides, prodrugs, active metabolites, and derivatives thereof;

q. AZD 4717 (available from NicOx S.A.) or acids, salts, enantiomers, analogs, esters, amides, prodrugs, active metabolites, and derivatives thereof;

r. NCX 1510/NCX 1512 (available from NicOx S.A.) or acids, salts, enantiomers, analogs, esters, amides, prodrugs, active metabolites, and derivatives thereof;

s. NCX 2216 (available from NicOx S.A.) or acids, salts, enantiomers, analogs, esters, amides, prodrugs, active metabolites, and derivatives thereof;

t. NCX 4040 (available from NicOx S.A.) or acids, salts, enantiomers, analogs, esters, amides, prodrugs, active metabolites, and derivatives thereof;

u. Nitric oxide donors as disclosed in U.S. Pat. No. 5,155,137 or acids, salts, enantiomers, analogs, esters, amides, prodrugs, active metabolites, and derivatives thereof;

v. Nitric oxide donors as disclosed in U.S. Pat. No. 5,366,997 or acids, salts, enantiomers, analogs, esters, amides, prodrugs, active metabolites, and derivatives thereof;

w. Nitric oxide donors as disclosed in U.S. Pat. No. 5,405,919 or acids, salts, enantiomers, analogs, esters, amides, prodrugs, active metabolites, and derivatives thereof;

x. Nitric oxide donors as disclosed in U.S. Pat. No. 5,650,442 or acids, salts, enantiomers, analogs, esters, amides, prodrugs, active metabolites, and derivatives thereof;

y. Nitric oxide donors as disclosed in U.S. Pat. No. 5,700,830 or acids, salts, enantiomers, analogs, esters, amides, prodrugs, active metabolites, and derivatives thereof;

z. Nitric oxide donors as disclosed in U.S. Pat. No. 5,632,981 or acids, salts, enantiomers, analogs, esters, amides, prodrugs, active metabolites, and derivatives thereof;

aa. Nitric oxide donors as disclosed in U.S. Pat. No. 6,290,981 or acids, salts, enantiomers, analogs, esters, amides, prodrugs, active metabolites, and derivatives thereof;

bb. Nitric oxide donors as disclosed in U.S. Pat. No. 5,691,423 or acids, salts, enantiomers, analogs, esters, amides, prodrugs, active metabolites, and derivatives thereof;

cc. Nitric oxide donors as disclosed in U.S. Pat. No. 5,721,365 or acids, salts, enantiomers, analogs, esters, amides, prodrugs, active metabolites, and derivatives thereof;

dd. Nitric oxide donors as disclosed in U.S. Pat. No. 5,714,511 or acids, salts, enantiomers, analogs, esters, amides, prodrugs, active metabolites, and derivatives thereof;

ee. Nitric oxide donors as disclosed in U.S. Pat. No. 6,511,911 or acids, salts, enantiomers, analogs, esters, amides, prodrugs, active metabolites, and derivatives thereof; and

ff. Nitric oxide donors as disclosed in U.S. Pat. No. 5,814,666.

The identification of further compounds that have nitric oxide donor activity and would therefore be useful in the present invention can be determined by release profile and/or induced vasospasm studies as described in U.S. Pat. Nos. 6,451,337 and 6,358,536, as well as Moon (2002) IBJU Int. 89: 942-9 and Fathian-Sabet et al. (2001) J. Urol. 165: 1724-9.

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species.

As used herein, treating and treatment refer to a reduction in at least one symptom selected from urinary frequency, urinary urgency, urinary urge incontinence, nocturia and enuresis, which is associated with lower urinary tract disorder.

As used herein, therapeutically effective amount refers to an amount sufficient to elicit the desired biological response. In the present invention, the desired biological response is a reduction (complete or partial) of at least one symptom associated with the lower urinary tract disorder being treated wherein the symptom is selected from urinary frequency, urinary urgency, urinary urge incontinence, nocturia and enuresis. As with any treatment, particularly treatment of a multi-symptom disorder, for example, overactive bladder, it is advantageous to treat as many disorder-related symptoms which the subject experiences.

A therapeutically effective amount can be achieved in the method of the invention employing a first amount of an α₂δ subunit calcium channel ligand and a second amount of a substituted aminomethyl-phenyl-cyclohexane derivative. In one embodiment, the α₂δ subunit calcium channel ligand and substituted aminomethyl-phenyl-cyclohexane derivative are each administered in a therapeutically effective amount (i.e., each in an amount which would be therapeutically effective if administered alone). In another embodiment, the α₂δ subunit calcium channel ligand and substituted aminomethyl-phenyl-cyclohexane derivative are each administered in an amount which alone does not provide a therapeutic effect (a sub-therapeutic dose). In yet another embodiment, the α₂δ subunit calcium channel ligand can be administered in a therapeutically effective amount, while the substituted aminomethyl-phenyl-cyclohexane derivative is administered in a sub-therapeutic dose. In still another embodiment, the α₂δ subunit calcium channel ligand can be administered in a sub-therapeutic dose, while the substituted aminomethyl-phenyl-cyclohexane derivative is administered in a therapeutically effective amount. It is understood that the method of coadministration of a first amount of an α₂δ subunit calcium channel ligand and a second amount of a substituted aminomethyl-phenyl-cyclohexane derivative can result in an enhanced or synergistic therapeutic effect, wherein the combined effect is greater than the additive effect that would result from separate administration of the first amount of the α₂δ subunit calcium channel ligand and the second amount of the substituted aminomethyl-phenyl-cyclohexane derivative.

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

Pharmaceutically acceptable carrier, includes pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Pharmaceutically acceptable carriers can be aqueous or non-aqueous solvents. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media.

### Modes of Administration

The compounds for use in the methods, pharmaceutical compositions or kits of the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intraduodenal, intrathecal, subcutaneous, intramuscular, intradermal, intraarterial, intravenous, inhalation, and topical administration.

Suitable compositions and dosage forms include tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. Further, those of ordinary skill in the art can readily deduce that suitable formulations involving these compositions and dosage forms, including those formulations as described elsewhere herein.

The term intravesical administration is used herein in its conventional sense to mean delivery of a drug directly into the bladder.

For oral administration the compounds can be in a suitable oral dosage form, such as tablets, capsules or caplets prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated, e.g., to provide for ease of swallowing or to provide a delayed release of active, using suitable methods. Liquid preparation for oral administration can be in the form of solutions, syrups or suspensions. Liquid preparations (e.g., solutions, suspensions and syrups) are also suitable for oral administration and can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and

preservatives (e.g., methyl or propyl p-hydroxy benzoates or sorbic acid).

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

The dosage form may also be a capsule, in which case the active agent-containing composition may be encapsulated in the form of a liquid or solid (including particulates such as granules, beads, powders or pellets). Suitable capsules can be hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals. If the active agent-containing composition is present within the capsule in liquid form, a liquid carrier can be used to dissolve the active agent(s). The carrier should be compatible with the capsule material and all components of the pharmaceutical composition, and should be suitable for ingestion.

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

Preferred buccal dosage forms will typically comprise a therapeutically effective amount of an active agent and a bioerodible (hydrolyzable) polymeric carrier that may also serve to adhere the dosage form to the buccal mucosa. The buccal dosage unit can be fabricated so as to erode over a predetermined time period, wherein drug delivery is provided essentially throughout. The time period is typically in the range of from about 1 hour to about 72 hours. Preferred buccal delivery preferably occurs over a time period of from about 2 hours to about 24 hours. Buccal drug delivery for short term use should preferably occur over a time period of from about 2 hours to about 8 hours, more preferably over a time period of from about 3 hours to about 4 hours. As needed buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The amount of the active agent in the buccal dosage unit will of course depend on the potency of the agent and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with the active agents to be administered and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and copolymers, e.g., those known as “carbomers” (Carbopol®, which may be obtained from B.F. Goodrich, is one such polymer). Other suitable polymers include, but are not limited to: hydrolyzed polyvinylalcohol; polyethylene oxides (e.g., Sentry Polyox® water soluble resins, available from Union Carbide); polyacrylates (e.g., Gantrez®, which may be obtained from GAF); vinyl polymers and copolymers; polyvinylpyrrolidone; dextran; guar gum; pectins; starches; and cellulosic polymers such as hydroxypropyl methylcellulose, (e.g., Methocel®, which may be obtained from the Dow Chemical Company), hydroxypropyl cellulose (e.g., Klucel®, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components can also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone® XL, which may be obtained from GAF), cross-linked carboxylic methylcelluloses, such as croscarmelose (e.g., Ac-di-sol®, which may be obtained from FMC), alginic acid, and sodium carboxymethyl starches (e.g., Explotab®, which can be obtained from Edward Medell Co., Inc.), methylcellulose, agar bentonite and alginic acid. Suitable diluents include those which are generally useful in pharmaceutical formulations prepared using compression techniques, e.g., dicalcium phosphate dihydrate (e.g., Di-Tab®, which may be obtained from Stauffer), sugars that have been processed by cocrystallization with dextrin (e.g., co-crystallized sucrose and dextrin such as Di-Pak®, which may be obtained from Amstar), calcium phosphate, cellulose, kaolin, mannitol, sodium chloride, dry starch, powdered sugar and the like. Binders, if used, include those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include tablets, creams, ointments, lozenges, pastes, and any other suitable dosage form where the active ingredient is admixed into a disintegrable matrix. The tablet, cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units can be fabricated to disintegrate rapidly. The time period for complete disintegration of the dosage unit is typically in the range of from about 10 seconds to about 30 minutes, and optimally is less than 5 minutes.

Other components can also be incorporated into the sublingual and lingual dosage forms described herein. The additional components include, but are not limited to binders, disintegrants, wetting agents, lubricants, and the like. Examples of binders that can be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy, supra).

With regard to transurethal administration, a transurethral permeation enhance can be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone® from Nelson Research & Development Co., Irvine, Calif.), SEPA® (available from Macrochem Co., Lexington, Mass.), surfactants as discussed above, including, for example, Tergitol®, Nonoxynol-9® and TWEEN-80®, and lower alkanols such as ethanol.

Transurethral drug administration, as explained in U.S. Pat. Nos. 5,242,391, 5,474,535, 5,686,093 and 5,773,020, can be carried out in a number of different ways using a variety of urethral dosage forms. For example, the drug can be introduced into the urethra from a flexible tube, squeeze bottle, pump or aerosol spray. The drug may also be contained in coatings, pellets or suppositories that are absorbed, melted or bioeroded in the urethra. In certain embodiments, the drug is included in a coating on the exterior surface of a penile insert. It is preferred, although not essential, that the drug be delivered from at least about 3 cm into the urethra, and preferably from at least about 7 cm into the urethra. Generally, delivery from at least about 3 cm to about 8 cm into the urethra will provide effective results in conjunction with the present method.

Urethral suppository formulations containing PEG or a PEG derivative can be conveniently formulated using conventional techniques, e.g., compression molding, heat molding or the like, as will be appreciated by those skilled in the art and as described in the pertinent literature and pharmaceutical texts. (See, e.g., Remington: The Science and Practice of Pharmacy, supra), which discloses typical methods of preparing pharmaceutical compositions in the form of urethral suppositories. The PEG or PEG derivative preferably has a molecular weight in the range of from about 200 to about 2,500 g/mol, more preferably in the range of from about 1,000 to about 2,000 g/mol. Suitable polyethylene glycol derivatives include polyethylene glycol fatty acid esters, for example, polyethylene glycol monostearate, polyethylene glycol sorbitan esters, e.g., polysorbates, and the like. Depending on the particular active agent, urethral suppositories may contain one or more solubilizing agents effective to increase the solubility of the active agent in the PEG or other transurethral vehicle.

It may be desirable to deliver the active agent in a urethral dosage form that provides for controlled or sustained release of the agent. In such a case, the dosage form can comprise a biocompatible, biodegradable material, typically a biodegradable polymer. Examples of such polymers include polyesters, polyalkylcyanoacrylates, polyorthoesters, polyanhydrides, albumin, gelatin and starch. As explained, for example, in PCT Publication No. WO 96/40054, these and other polymers can be used to provide biodegradable microparticles that enable controlled and sustained drug release, in turn minimizing the required dosing frequency.

The urethral dosage form will preferably comprise a suppository that is from about 2 to about 20 mm in length, preferably from about 5 to about 10 mm in length, and less than about 5 mm in width, preferably less than about 2 mm in width. The weight of the suppository will typically be in the range of from about 1 mg to about 100 mg, preferably in the range of from about 1 mg to about 50 mg. However, it will be appreciated by those skilled in the art that the size of the suppository can and will vary, depending on the potency of the drug, the nature of the formulation, and other factors.

Transurethral drug delivery may involve an “active” delivery mechanism such as iontophoresis, electroporation or phonophoresis. Devices and methods for delivering drugs in this way are well known in the art. Iontophoretically assisted drug delivery is, for example, described in PCT Publication No. WO 96/40054, cited above. Briefly, the active agent is driven through the urethral wall by means of an electric current passed from an external electrode to a second electrode contained within or affixed to a urethral probe.

Preferred transrectal dosage forms can include rectal suppositories, creams, ointments, and liquid formulations (enemas). The suppository, cream, ointment or liquid formulation for transrectal delivery comprises a therapeutically effective amount of the selected agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration is preferably in the range of from about 10 minutes to about 6 hours, and optimally is less than about 3 hours.

Other components can also be incorporated into the transrectal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

Preferred vaginal or perivaginal dosage forms include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration is preferably in the range of from about 10 minutes to about 6 hours, and optimally is less than about 3 hours.

Other components can also be incorporated into the vaginal or perivaginal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

The active agents can also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. Preferably, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations can be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation can also comprise dry powder formulations, particularly insufflations in which the powder has an average particle size of from about 0.1 μm to about 50 μm, preferably from about 1 μm to about 25 μm.

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with lower urinary tract disorders.

Another system available from Medtronic that is commonly utilized for intrathecal administration is the fully implantable, programmable SynchroMed® Infusion System. The SynchroMed® Infusion System has two parts that are both placed in the body during a surgical procedure: the catheter and the pump. The catheter is a small, soft tube. One end is connected to the catheter port of the pump, and the other end is placed in the intrathecal space. The pump is a round metal device about one inch (2.5 cm) thick, three inches (8.5 cm) in diameter, and weighs about six ounces (205 g) that stores and releases prescribed amounts of medication directly into the intrathecal space. It can be made of titanium, a lightweight, medical-grade metal. The reservoir is the space inside the pump that holds the medication. The fill port is a raised center portion of the pump through which the pump is refilled. The doctor or a nurse inserts a needle through the patient's skin and through the fill port to fill the pump. Some pumps have a side catheter access port that allows the doctor to inject other medications or sterile solutions directly into the catheter, bypassing the pump.

The SynchroMed® pump automatically delivers a controlled amount of medication through the catheter to the intrathecal space around the spinal cord, where it is most effective. The exact dosage, rate and timing prescribed by the doctor are entered in the pump using a programmer, an external computer-like device that controls the pump's memory. Information about the patient's prescription can be stored in the pump's memory. The doctor can easily review this information by using the programmer. The programmer communicates with the pump by radio signals that allow the doctor to tell how the pump is operating at any given time. The doctor also can use the programmer to change your medication dosage.

Methods of intrathecal administration can include those described above available from Medtronic, as well as other methods that are known to one of skill in the art.

Suitable methods for intravesical administration can be found in U.S. Pat. Nos. 6,207,180 and 6,039,967.

For other parenteral administration, the compounds for use in the method of the invention can be formulated for injection or infusion, for example, intravenous, intra-arterial, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

### Additional Dosage Formulations and Drug Delivery Systems

As compared with traditional drug delivery approaches, some controlled release technologies rely upon the modification of both macromolecules and synthetic small molecules to allow them to be actively instead of passively absorbed into the body. For example, XenoPort Inc. utilizes technology that takes existing molecules and re-engineers them to create new chemical entities (unique molecules) that have improved pharmacologic properties to either: 1) lengthen the short half-life of a drug; 2) overcome poor absorption; and/or 3) deal with poor drug distribution to target tissues. Techniques to lengthen the short half-life of a drug include the use of prodrugs with slow cleavage rates to release drugs over time or that engage transporters in small and large intestines to allow the use of oral sustained delivery systems, as well as drugs that engage active transport systems. Examples of such controlled release formulations, tablets, dosage forms, and drug delivery systems, and that are suitable for use with the present invention, are described in the following published US and PCT patent applications assigned to Xenoport Inc.: US20030158254; US20030158089; US20030017964; US2003130246; WO02100172; WO02100392; WO02100347; WO02100344; WO0242414; WO0228881; WO0228882; WO0244324; WO0232376; WO0228883; and WO0228411. In particular, Xenoport's XP13512 is a transported Prodrug of gabapentin that has been engineered to utilize high capacity transport mechanisms located in both the small and large intestine and to rapidly convert to gabapentin once in the body. In contrast to gabapentin itself, XP13512 was shown in preclinical and clinical studies to produce dose proportional blood levels of gabapentin across a broad range of oral doses, and to be absorbed efficiently from the large intestine.

Some other controlled release technologies rely upon methods that promote or enhance gastric retention, such as those developed by Depomed Inc. Because many drugs are best absorbed in the stomach and upper portions of the small intestine, Depomed has developed tablets that swell in the stomach during the postprandial or fed mode so that they are treated like undigested food. These tablets therefore sit safely and neutrally in the stomach for 6, 8, or more hours and deliver drug at a desired rate and time to upper gastrointestinal sites. Specific technologies in this area include: 1) tablets that slowly erode in gastric fluids to deliver drugs at almost a constant rate (particularly useful for highly insoluble drugs); 2) bi-layer tablets that combine drugs with different characteristics into a single table (such as a highly insoluble drug in an erosion layer and a soluble drug in a diffusion layer for sustained release of both); and 3) combination tablets that can either deliver drugs simultaneously or in sequence over a desired period of time (including an initial burst of a fast acting drug followed by slow and sustained delivery of another drug). Examples of such controlled release formulations that are suitable for use with the present invention and that rely upon gastric retention during the postprandial or fed mode, include tablets, dosage forms, and drug delivery systems in the following US patents assigned to Depomed Inc.: U.S. Pat. Nos. 6,488,962; 6,451,808; 6,340,475; 5,972,389; 5,582,837; and 5,007,790. Examples of such controlled release formulations that are suitable for use with the present invention and that rely upon gastric retention during the postprandial or fed mode, include tablets, dosage forms, and drug delivery systems in the following published US and PCT patent applications assigned to Depomed Inc.: US20030147952; US20030104062; US20030104053; US20030104052; US20030091630; US20030044466; US20030039688; US20020051820; WO0335040; WO0335039; WO0156544; WO0132217; WO9855107; WO9747285; and WO9318755.

Other controlled release systems include those developed by ALZA Corporation based upon: 1) osmotic technology for oral delivery; 2) transdermal delivery via patches; 3) liposomal delivery via intravenous injection; 4) osmotic technology for long-term delivery via implants; and 5) depot technology designed to deliver agents for periods of days to a month. ALZA oral delivery systems include those that employ osmosis to provide precise, controlled drug delivery for up to 24 hours for both poorly soluble and highly soluble drugs, as well as those that deliver high drug doses meeting high drug loading requirements. ALZA controlled transdermal delivery systems provide drug delivery through intact skin for as long as one week with a single application to improve drug absorption and deliver constant amounts of drug into the bloodstream over time. ALZA liposomal delivery systems involve lipid nanoparticles that evade recognition by the immune system because of their unique polyethylene glycol (PEG) coating, allowing the precise delivery of drugs to disease-specific areas of the body. ALZA also has developed osmotically driven systems to enable the continuous delivery of small drugs, peptides, proteins, DNA and other bioactive macromolecules for up to one year for systemic or tissue-specific therapy. Finally, ALZA depot injection therapy is designed to deliver biopharmaceutical agents and small molecules for periods of days to a month using a nonaqueous polymer solution for the stabilization of macromolecules and a unique delivery profile.

Examples of controlled release formulations, tablets, dosage forms, and drug delivery systems that are suitable for use with the present invention are described in the following US patents assigned to ALZA Corporation: U.S. Pat. Nos. 4,367,741; 4,402,695; 4,418,038; 4,434,153; 4,439,199; 4,450,198; 4,455,142; 4,455,144; 4,484,923; 4,486,193; 4,489,197; 4,511,353; 4,519,801; 4,526,578; 4,526,933; 4,534,757; 4,553,973; 4,559,222; 4,564,364; 4,578,075; 4,588,580; 4,610,686; 4,612,008; 4,618,487; 4,627,851; 4,629,449; 4,642,233; 4,649,043; 4,650,484; 4,659,558; 4,661,105; 4,662,880; 4,675,174; 4,681,583; 4,684,524; 4,692,336; 4,693,895; 4,704,119; 4,705,515; 4,717,566; 4,721,613; 4,723,957; 4,725,272; 4,728,498; 4,743,248; 4,747,847; 4,751,071; 4,753,802; 4,755,180; 4,756,314; 4,764,380; 4,773,907; 4,777,049; 4,781,924; 4,783,337; 4,786,503; 4,788,062; 4,810,502; 4,812,313; 4,816,258; 4,824,675; 4,834,979; 4,837,027; 4,842,867; 4,846,826; 4,847,093; 4,849,226; 4,851,229; 4,851,231; 4,851,232; 4,853,229; 4,857,330; 4,859,470; 4,863,456; 4,863,744; 4,865,598; 4,867,969; 4,871,548; 4,872,873; 4,874,388; 4,876,093; 4,892,778; 4,902,514; 4,904,474; 4,913,903; 4,915,949; 4,915,952; 4,917,895; 4,931,285; 4,946,685; 4,948,592; 4,954,344; 4,957,494; 4,960,416; 4,961,931; 4,961,932; 4,963,141; 4,966,769; 4,971,790; 4,976,966; 4,986,987; 5,006,346; 5,017,381; 5,019,397; 5,023,076; 5,023,088; 5,024,842; 5,028,434; 5,030,454; 5,071,656; 5,077,054; 5,082,668; 5,104,390; 5,110,597; 5,122,128; 5,125,894; 5,141,750; 5,141,752; 5,156,850; 5,160,743; 5,160,744; 5,169,382; 5,171,576; 5,176,665; 5,185,158; 5,190,765; 5,198,223; 5,198,229; 5,200,195; 5,200,196; 5,204,116; 5,208,037; 5,209,746; 5,221,254; 5,221,278; 5,229,133; 5,232,438; 5,232,705; 5,236,689; 5,236,714; 5,240,713; 5,246,710; 5,246,711; 5,252,338; 5,254,349; 5,266,332; 5,273,752; 5,284,660; 5,286,491; 5,308,348; 5,318,558; 5,320,850; 5,322,502; 5,326,571; 5,330,762; 5,338,550; 5,340,590; 5,342,623; 5,344,656; 5,348,746; 5,358,721; 5,364,630; 5,376,377; 5,391,381; 5,402,777; 5,403,275; 5,411,740; 5,417,675; 5,417,676; 5,417,682; 5,423,739; 5,424,289; 5,431,919; 5,443,442; 5,443,459; 5,443,461; 5,456,679; 5,460,826; 5,462,741; 5,462,745; 5,489,281; 5,499,979; 5,500,222; 5,512,293; 5,512,299; 5,529,787; 5,531,736; 5,532,003; 5,533,971; 5,534,263; 5,540,912; 5,543,156; 5,571,525; 5,573,503; 5,591,124; 5,593,695; 5,595,759; 5,603,954; 5,607,696; 5,609,885; 5,614,211; 5,614,578; 5,620,705; 5,620,708; 5,622,530; 5,622,944; 5,633,011; 5,639,477; 5,660,861; 5,667,804; 5,667,805; 5,674,895; 5,688,518; 5,698,224; 5,702,725; 5,702,727; 5,707,663; 5,713,852; 5,718,700; 5,736,580; 5,770,227; 5,780,058; 5,783,213; 5,785,994; 5,795,591; 5,811,465; 5,817,624; 5,824,340; 5,830,501; 5,830,502; 5,840,754; 5,858,407; 5,861,439; 5,863,558; 5,876,750; 5,883,135; 5,840,754; 5,897,878; 5,904,934; 5,904,935; 5,906,832; 5,912,268; 5,914,131; 5,916,582; 5,932,547; 5,938,654; 5,941,844; 5,955,103; 5,972,369; 5,972,370; 5,972,379; 5,980,943; 5,981,489; 5,983,130; 5,989,590; 5,995,869; 5,997,902; 6,001,390; 6,004,309; 6,004,578; 6,008,187; 6,020,000; 6,034,101; 6,036,973; 6,039,977; 6,057,374; 6,066,619; 6,068,850; 6,077,538; 6,083,190; 6,096,339; 6,106,845; 6,110,499; 6,120,798; 6,120,803; 6,124,261; 6,124,355; 6,130,200; 6,146,662; 6,153,678; 6,174,547; 6,183,466; 6,203,817; 6,210,712; 6,210,713; 6,224,907; 6,235,712; 6,245,357; 6,262,115; 6,264,990; 6,267,984; 6,287,598; 6,289,241; 6,331,311; 6,333,050; 6,342,249; 6,346,270; 6,365,183; 6,368,626; 6,387,403; 6,419,952; 6,440,457; 6,468,961; 6,491,683; 6,512,010; 6,514,530; 6534089; 6,544,252; 6,548,083; 6,551,613; 6,572,879; and 6,596,314

Other examples of controlled release formulations, tablets, dosage forms, and drug delivery systems that are suitable for use with the present invention are described in the following published US patent application and PCT applications assigned to ALZA Corporation: US20010051183; WO0004886; WO0013663; WO0013674; WO0025753; WO0025790; WO0035419; WO0038650; WO0040218; WO0045790; WO0066126; WO0074650; WO019337; WO0119352; WO0121211; WO0137815; WO0141742; WO0143721; WO0156543; WO3041684; WO03041685; WO03041757; WO03045352; WO03051341; WO03053400; WO03053401; WO9000416; WO9004965; WO9113613; WO9116884; WO9204011; WO9211843; WO9212692; WO9213521; WO9217239; WO9218102; WO9300071; WO9305843; WO9306819; WO9314813; WO9319739; WO9320127; WO9320134; WO9407562; WO9408572; WO9416699; WO9421262; WO9427587; WO9427589; WO9503823; WO9519174; WO9529665; WO9600065; WO9613248; WO9625922; WO9637202; WO9640049; WO9640050; WO9640139; WO9640364; WO9640365; WO9703634; WO9800158; WO9802169; WO9814168; WO9816250; WO9817315; WO9827962; WO9827963; WO9843611; WO9907342; WO9912526; WO9912527; WO9918159; WO9929297; WO9929348; WO9932096; WO9932153; WO9948494; WO9956730; WO9958115; and WO9962496.

Another drug delivery technology suitable for use in the present invention is that disclosed by DepoMed, Inc. in U.S. Pat. No. 6,682,759, which discloses a method for manufacturing a pharmaceutical tablet for oral administration combining both immediate-release and prolonged-release modes of drug delivery. The tablet according to the method comprises a prolonged-release drug core and an immediate-release drug coating or layer, which can be insoluble or sparingly soluble in water. The method limits the drug particle diameter in the immediate-release coating or layer to 10 microns or less. The coating or layer is either the particles themselves, applied as an aqueous suspension, or a solid composition that contains the drug particles incorporated in a solid material that disintegrates rapidly in gastric fluid.

Andrx Corporation has also developed drug delivery technology suitable for use in the present invention that includes: 1) a pelletized pulsatile delivery system (“PPDS”); 2) a single composition osmotic tablet system (“SCOT”); 3) a solubility modulating hydrogel system (“SMHS”); 4) a delayed pulsatile hydrogel system (“DPHS”); 5) a stabilized pellet delivery system (“SPDS”); 6) a granulated modulating hydrogel system (“GMHS”); 7) a pelletized tablet system (“PELTAB”); 8) a porous tablet system (“PORTAB”); and 9) a stabilized tablet delivery system (“STDS”). PPDS uses pellets that are coated with specific polymers and agents to control the release rate of the microencapsulated drug and is designed for use with drugs that require a pulsed release. SCOT utilizes various osmotic modulating agents as well as polymer coatings to provide a zero-order drug release. SMHS utilizes a hydrogel-based dosage system that avoids the “initial burst effect” commonly observed with other sustained-release hydrogel formulations and that provides for sustained release without the need to use special coatings or structures that add to the cost of manufacturing. DPHS is designed for use with hydrogel matrix products characterized by an initial zero-order drug release followed by a rapid release that is achieved by the blending of selected hydrogel polymers to achieve a delayed pulse. SPDS incorporates a pellet core of drug and protective polymer outer layer, and is designed specifically for unstable drugs, while GMHS incorporates hydrogel and binding polymers with the drug and forms granules that are pressed into tablet form. PELTAB provides controlled release by using a water insoluble polymer to coat discrete drug crystals or pellets to enable them to resist the action of fluids in the gastrointestinal tract, and these coated pellets are then compressed into tablets. PORTAB provides controlled release by incorporating an osmotic core with a continuous polymer coating and a water soluble component that expands the core and creates microporous channels through which drug is released. Finally, STDS includes a dual layer coating technique that avoids the need to use a coating layer to separate the enteric coating layer from the omeprazole core.

Examples of controlled release formulations, tablets, dosage forms, and drug delivery systems that are suitable for use with the present invention are described in the following US patents assigned to Andrx Corporation: U.S. Pat. Nos. 5,397,574; 5,419,917; 5,458,887; 5,458,888; 5,472,708; 5,508,040; 5,558,879; 5,567,441; 5,654,005; 5,728,402; 5,736,159; 5,830,503; 5,834,023; 5,837,379; 5,916,595; 5,922,352; 6,099,859; 6,099,862; 6,103,263; 6,106,862; 6,156,342; 6,177,102; 6,197,347; 6,210,716; 6,238,703; 6,270,805; 6,284,275; 6,485,748; 6,495,162; 6,524,620; 6,544,556; 6,589,553; 6,602,522; and 6,610,326.

Examples of controlled release formulations, tablets, dosage forms, and drug delivery systems that are suitable for use with the present invention are described in the following published US and PCT patent applications assigned to Andrx Corporation: US20010024659; US20020115718; US20020156066; WO0004883; WO0009091; WO0012097; WO0027370; WO0050010; WO0132161; WO0134123; WO0236077; WO0236100; WO02062299; WO02062824; WO02065991; WO02069888; WO02074285; WO03000177; WO9521607; WO9629992; WO9633700; WO9640080; WO9748386; WO9833488; WO9833489; WO9930692; WO9947125; and WO9961005.

Some other examples of drug delivery approaches focus on non-oral drug delivery, providing parenteral, transmucosal, and topical delivery of proteins, peptides, and small molecules. For example, the Atrigel® drug delivery system marketed by Atrix Laboratories Inc. comprises biodegradable polymers, similar to those used in biodegradable sutures, dissolved in biocompatible carriers. These pharmaceuticals may be blended into a liquid delivery system at the time of manufacturing or, depending upon the product, may be added later by a physician at the time of use. Injection of the liquid product subcutaneously or intramuscularly through a small gauge needle, or placement into accessible tissue sites through a cannula, causes displacement of the carrier with water in the tissue fluids, and a subsequent precipitate to form from the polymer into a solid film or implant. The drug encapsulated within the implant is then released in a controlled manner as the polymer matrix biodegrades over a period ranging from days to months. Examples of such drug delivery systems include Atrix's Eligard®, Atridox®/Doxirobe®, Atrisorb® FreeFlow™/Atrisorb®-D FreeFlow, bone growth products, and others as described in the following published US and PCT patent applications assigned to Atrix Laboratories Inc.: U.S. Pat. No. Re37950; U.S. Pat. Nos. 6,630,155; 6,566,144; 6,610,252; 6,565,874; 6,528,080; 6,461,631; 6,395,293; 6,261,583; 6,143,314; 6,120,789; 6,071,530; 5,990,194; 5,945,115; 5,888,533; 5,792,469; 5,780,044; 5,759,563; 5,744,153; 5,739,176; 5,736,152; 5,733,950; 5,702,716; 5,681,873; 5,660,849; 5,599,552; 5,487,897; 5,368,859; 5,340,849; 5,324,519; 5,278,202; 5,278,201; US20020114737, US20030195489; US20030133964; US20010042317; US20020090398; US20020001608; and US2001042317.

Atrix Laboratories Inc. also markets technology for the non-oral transmucosal delivery of drugs over a time period from minutes to hours. For example, Atrix's BEMA™ (Bioerodible Muco-Adhesive Disc) drug delivery system comprises pre-formed bioerodible discs for local or systemic delivery. Examples of such drug delivery systems include those as described in U.S. Pat. No. 6,245,345. Other drug delivery systems marketed by Atrix Laboratories Inc. focus on topical drug delivery. For example, SMP™ (Solvent Particle System) allows the topical delivery of highly water-insoluble drugs. This product allows for a controlled amount of a dissolved drug to permeate the epidermal layer of the skin by combining the dissolved drug with a microparticle suspension of the drug. The SMP™ system works in stages whereby: 1) the product is applied to the skin surface; 2) the product near follicles concentrates at the skin pore; 3) the drug readily partitions into skin oils; and 4) the drug diffuses throughout the area. By contrast, MCA® (Mucocutaneous Absorption System) is a water-resistant topical gel providing sustained drug delivery. MCA® forms a tenacious film for either wet or dry surfaces where: 1) the product is applied to the skin or mucosal surface; 2) the product forms a tenacious moisture-resistant film; and 3) the adhered film provides sustained release of drug for a period from hours to days. Yet another product, BCP™ (Biocompatible Polymer System) provides a non-cytotoxic gel or liquid that is applied as a protective film for wound healing. Examples of these systems include Orajel®-Ultra Mouth Sore Medicine as well as those as described in the following published US patents and applications assigned to Atrix Laboratories Inc.: U.S. Pat. Nos. 6,537,565; 6,432,415; 6,355,657; 5,962,006; 5,725,491; 5,722,950; 5,717,030; 5,707,647; 5,632,727; and US20010033853.

Additional formulations and compositions available from Teva Pharmaceutical Industries Ltd., Warner Lambert & Co., and Godecke Aktiengesellshaft that include gabapentin and are useful in the present invention include those as described in the following US patents and published US and PCT patent applications: U.S. Pat. Nos. 6,531,509; 6,255,526; 6,054,482; US2003055109; US2002045662; US2002009115; WO 01/97782; WO 01/97612; EP 2001946364; WO 99/59573; and WO 99/59572.

### Topical Formulations

Topical formulations can be in any form suitable for application to the body surface, and may comprise, for example, an ointment, cream, gel, lotion, solution, paste or the like, and/or may be prepared so as to contain liposomes, micelles, and/or microspheres. Preferred topical formulations herein are ointments, creams and gels.

Ointments, as is well known in the art of pharmaceutical formulation, are semisolid preparations that are typically based on petrolatum or other petroleum derivatives. The specific ointment base to be used, preferably provides for optimum drug delivery, and, preferably, will provides for other desired characteristics as well, e.g., emolliency or the like. The ointment base is preferably inert, stable, nonirritating and nonsensitizing. As explained in Remington: The Science and Practice of Pharmacy, supra, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy, supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels-are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

### Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. The drug-containing reservoir and skin contact adhesive can also be separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed above in transmucosal compositions.

The formulations of the present invention can be, but are not limited to, short-term, rapid-offset, controlled, for example, sustained release, delayed release and pulsatile release formulations.

The term sustained release is used in its conventional sense to refer to a drug formulation that provides for gradual release of a drug over an extended period of time, and that preferably, although not necessarily, results in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles for example, by injection or in the form of wafers or discs by implantation.

The term delayed release is used herein in its conventional sense to refer to a drug formulation that provides for an initial release of the drug after some delay following drug administration and that preferably, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

### Coadministration

In practicing the methods of the invention, coadministration refers to administration of a first amount of an α₂δ subunit calcium channel ligand and a second amount of a substituted aminomethyl-phenyl-cyclohexane derivative, wherein the first and second amounts together comprise a therapeutically effective amount to treat at least one symptom of a lower urinary tract disorder in a subject in need of treatment, wherein the symptom is selected from the group consisting of urinary frequency, urinary urgency, urinary urge incontinence, nocturia and enuresis. Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When coadministration involves the separate administration of the first amount of an α₂δ subunit calcium channel ligand and the second amount of the substituted aminomethyl-phenyl-cyclohexane derivative the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, the α₂δ subunit calcium channel ligand and the substituted aminomethyl-phenyl-cyclohexane derivative can be administered in any order within about 24 hours of each other, within about 16 hours of each other, within about 8 hours of each other, within about 4 hours of each other, within about 1 hour of each other or within about 30 minutes of each other.

### Dosing

The therapeutically effective amount of a first amount of an α₂δ subunit calcium channel ligand and a second amount of a substituted aminomethyl-phenyl-cyclohexane derivative in combination will depend on the age, sex and weight of the patient, the current medical condition of the patient and the nature of the lower urinary tract disorder being treated. The skilled artisan will be able to determine appropriate dosages depending on these and other factors.

As used herein, continuous dosing refers to the chronic administration of a selected active agent.

As used herein, as-needed dosing, also known as “pro re nata” “pm” dosing, and “on demand” dosing or administration is meant the administration of a therapeutically effective dose of the compound(s) at some time prior to commencement of an activity wherein suppression of a lower urinary tract disorder would be desirable.

Administration can be immediately prior to such an activity, including about 0 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, or about 10 hours prior to such an activity, depending on the formulation.

In a particular embodiment, drug administration or dosing is on an as-needed basis, and does not involve chronic drug administration. With an immediate release dosage form, as-needed administration can involve drug administration immediately prior to commencement of an activity wherein suppression of the symptoms of overactive bladder would be desirable, but will generally be in the range of from about 0 minutes to about 10 hours prior to such an activity, preferably in the range of from about 0 minutes to about 5 hours prior to such an activity, most preferably in the range of from about 0 minutes to about 3 hours prior to such an activity.

A suitable dose per day of the α₂δ subunit calcium channel ligand or substituted aminomethyl-phenyl-cyclohexane derivative for administration can be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg.

Other suitable doses per day of the α₂δ subunit calcium channel ligand or substituted aminomethyl-phenyl-cyclohexane derivative for administration include doses of about or greater than 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, about 500 μg (0.5 mg), about 1 mg, about 1.25 mg, about 1.5 mg, about 2.0 mg, about 2.5 mg, about 3.0 mg, about 3.5 mg, about 4.0 mg, about 4.5 mg, about S mg, about 10 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg.

In some instances, a dose suitable per day for intrathecal administration can be in the range of from about 1 fg to about 1 mg, about 5 fg to about 500 μg, about 10 fg to about 400 μg, about 20 fg to about 300 μg, about 30 fg to about 200 μg, about 40 fg to about 100 μg, about 50 fg to about 50 μg, about 100 fg to about 40 μg, about 200 fg to about 30 μg, about 300 fg to about 20 μg, about 400 fg to about 10 μg, about 500 fg to about 5 μg, about 1 pg to about 1 μg, about 5 pg to about 500 ng, about 10 pg to about 400 ng, about 20 pg to about 300 ng, about 30 pg to about 200 ng, about 40 pg to about 100 ng, about 50 pg to about 50 ng, about 100 pg to about 40 ng, about 200 pg to about 30 ng, about 300 pg to about 20 ng, about 400 pg to about 10 ng, about 500 pg to about 5 ng.

Other suitable doses per day of the α₂δ subunit calcium channel ligand or substituted aminomethyl-phenyl-cyclohexane derivative for certain intrathecal administrations include doses equal to or greater than about 1 fg, about 5 fg, about 10 fg, about 20 fg, about 30 fg, about 40 fg, about 50 fg, about 100 fg, about 200 fg, about 300 fg, about 400 fg, about 500 fg, about 1 pg, about 5 pg, about 10 pg, about 20 pg, about 30 pg, about 40 pg, about 50 pg, about 100 pg, about 200 pg, about 300 pg, about 400 pg, about 500 pg, about 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, or about 500 μg.

In a particular embodiment, the dose of α₂δ subunit calcium channel ligand can be in the range of from about 50 mg to about 5000 mg per day, such as from about 100 mg to about 2500 mg, for example, from about 500 mg to about 2000 mg per day.

In a particular embodiment, the substituted aminomethyl-phenyl-cyclohexane derivative can be in the range of from about 0.20 mg to about 2000 mg per day, such as from about 1 mg to about 1000 mg, for example, from about 5 mg to about 500 mg, such as about 20 mg to about 400 mg per day.

It is understood that the dose can be administered in a single dosage or in multiple dosages, for example from 1 to 4 or more times per day. When multiple dosages are used, the amount of each dosage can be the same or different.

It is understood that a per day dose of the compounds of the combination can be administered every day, every other day, every 2 days, every 3 days, every 4 days, every 5 days etc. For example, with every other day administration a per day dose of both the α₂δ subunit calcium channel ligand and substituted aminomethyl-phenyl-cyclohexane derivative can be initiated on Monday with a first subsequent per day dose of both the α₂δ subunit calcium channel ligand and substituted aminomethyl-phenyl-cyclohexane derivative Wednesday, a second subsequent per day dose of both the α₂δ subunit calcium channel ligand and substituted aminomethyl-phenyl-cyclohexane derivative on Friday, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. Suitable amounts for use in preparation of a unit dosage form are described above for both the the α₂δ subunit calcium channel ligand and substituted aminomethyl-phenyl-cyclohexane derivative. The unit dosage form can be for a single daily dose or one of multiple daily doses (e.g., about 1 to 4 or more times per day). When multiple daily doses are used, the unit dosage form can be the same or different for each dose.

The invention further includes a kit for treating a lower urinary tract disorder. The kit comprises a compound which is an α₂δ subunit calcium channel ligand and instructions for use with a compound which is a substituted aminomethyl-phenyl-cyclohexane derivative, according to the method of the invention and optionally a device for administering the compounds of the invention. In a particular embodiment, the α₂δ subunit calcium channel ligand is present in the kit in a sub-therapeutic dose.

The invention further includes a kit for treating a lower urinary tract disorder. The kit comprises a compound which is a substituted aminomethyl-phenyl-cyclohexane derivative and instructions for use with a compound which is an α₂δ subunit calcium channel ligand, according to the method of the invention and optionally a device for administering the compounds of the invention. In a particular embodiment, the substituted aminomethyl-phenyl-cyclohexane derivative is present in the kit in a sub-therapeutic dose.

The invention further includes a kit for treating a lower urinary tract disorder. The kit comprises a first compound which is an α₂δ subunit calcium channel ligand, a second compound which is a substituted aminomethyl-phenyl-cyclohexane derivative and instructions for administering the first and second compounds, according to the method of the invention and optionally a device for administering the compounds of the invention. In a particular embodiment, at least one of the first or second compound is present in the kit in a sub-therapeutic dose.

Compounds can be in separate dosage forms or combined in a single dosage form. In other embodiments of the kits, the instructional insert further includes instructions for administration with an additional therapeutic agent as described herein.

It is understood that in practicing the method or using a kit of the present invention that administration encompasses administration by different individuals (e.g., the subject, physicians or other medical professionals) administering the same or different compounds.

As used herein, the term pharmaceutically acceptable salt refers to a salt of a compound to be administered prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

It is understood that suitable α₂δ subunit calcium channel ligands and substituted aminomethyl-phenyl-cyclohexane derivatives can be identified, for example, by screening libraries or collections of molecules using suitable methods. Another source for the compounds of interest are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods.

The invention also relates to a method of processing a claim under a health insurance policy submitted by a claimant seeking reimbursement for costs associated with the treatment of at least one symptom of a lower urinary tract disorder, wherein said treatment comprises administering a first amount of an α₂δ subunit calcium channel ligand and a second amount of a substituted aminomethyl-phenyl-cyclohexane derivative, wherein the first and second amounts together comprise a therapeutically effective amount comprising: reviewing said claim; determining whether said treatment is reimbursable under said insurance policy; and processing said claim to provide partial or complete reimbursement of said costs.

An “aliphatic group” is non-aromatic, consists solely of carbon and hydrogen and can optionally contain one or more units of unsaturation, e.g., double and/or triple bonds and/or one or more suitable substituents. An aliphatic group can be straight chained, branched or cyclic. When straight chained or branched, an aliphatic group typically contains between about 1 and about 12 carbon atoms, more typically between about 1 and about 6 carbon atoms. When cyclic, an aliphatic group typically contains between about 3 and about 10 carbon atoms, more typically between about 3 and about 7 carbon atoms. Aliphatic groups can be alkyl groups (i.e., completely saturated aliphatic groups), alkenyl groups (i.e., aliphatic groups having one or more carbon-carbon double bonds) or alkynyl groups (i.e., aliphatic groups having one or more carbon-carbon triple bonds). Aliphatic groups are preferably C₁-C₂ straight chained or branched alkyl groups (i.e., completely saturated aliphatic groups), more preferably C₁-C₆ straight chained or branched alkyl groups. Examples include methyl, ethyl, n-propyl, iso-propyl, n-butyl, sec-butyl and tert-butyl. Aliphatic groups can also be C₁-C₂ straight chained or branched alkenyl groups or alkynyl groups. Aliphatic groups can optionally be substituted with a designated number of substituents, as described herein.

An “aromatic group” (also referred to as an “aryl group”) as used herein includes carbocyclic aromatic groups, heterocyclic aromatic groups (also referred to as “heteroaryl”) and fused polycyclic aromatic ring systems as defined herein which can be optionally substituted with a suitable substituent.

A “carbocyclic aromatic group” is an aromatic ring of 5 to 14 carbons atoms, and includes a carbocyclic aromatic group fused with a 5-or 6-membered cycloalkyl group such as indan. Examples of carbocyclic aromatic groups include, but are not limited to, phenyl, naphthyl, e.g., 1-naphthyl and 2-naphthyl; anthracenyl, e.g., 1-anthracenyl, 2-anthracenyl; phenanthrenyl; fluorenonyl, e.g., 9-fluorenonyl, indanyl and the like. A carbocyclic aromatic group is optionally substituted with a designated number of substituents, described below.

A “heterocyclic aromatic group” (or “heteroaryl”) is a monocyclic, bicyclic or tricyclic aromatic ring of 5- to 14-ring atoms of carbon and from one to four heteroatoms selected from O, N, or S. Examples of heteroaryl include, but are not limited to pyridyl, e.g., 2-pyridyl (also referred to as α-pyridyl), 3-pyridyl (also referred to as β-pyridyl) and 4-pyridyl (also referred to as γ-pyridyl); thienyl, e.g., 2-thienyl and 3-thienyl; furanyl, e.g., 2-furanyl and 3-furanyl; pyrimidyl, e.g., 2-pyrimidyl and 4-pyrimidyl; imidazolyl, e.g., 2-imidazolyl; pyranyl, e.g., 2-pyranyl and 3-pyranyl; pyrazolyl, e.g., 4-pyrazolyl and 5-pyrazolyl; thiazolyl, e.g., 2-thiazolyl, 4-thiazolyl and 5-thiazolyl; thiadiazolyl; isothiazolyl; oxazolyl, e.g., 2-oxazoyl, 4-oxazoyl and 5-oxazoyl; isoxazoyl; pyrrolyl; pyridazinyl; pyrazinyl and the like. Heterocyclic aromatic (or heteroaryl) as defined above can be optionally substituted with a designated number of substituents, as described below for aromatic groups.

A “fused polycyclic aromatic” ring system is a carbocyclic aromatic group or heteroaryl fused with one or more other heteroaryl or nonaromatic heterocyclic ring. Examples include, quinolinyl and isoquinolinyl, e.g, 2-quinolinyl, 3-quinolinyl, 4-quinolinyl, 5-quinolinyl, 6-quinolinyl, 7-quinolinyl and 8-quinolinyl, 1-isoquinolinyl, 3-quinolinyl, 4-isoquinolinyl, 5-isoquinolinyl, 6-isoquinolinyl, 7-isoquinolinyl and 8-isoquinolinyl; benzofuranyl, e.g., 2-benzofuranyl and 3-benzofuranyl; dibenzofuranyl, e.g., 2,3-dihydrobenzofuranyl; dibenzothiophenyl; benzothienyl, e.g., 2-benzothienyl and 3-benzothienyl; indolyl, e.g., 2-indolyl and 3-indolyl; benzothiazolyl, e.g., 2-benzothiazolyl; benzooxazolyl, e.g., 2-benzooxazolyl; benzimidazolyl, e.g., 2-benzoimidazolyl; isoindolyl, e.g., 1-isoindolyl and 3-isoindolyl; benzotriazolyl; purinyl; thianaphthenyl and the like. Fused polycyclic aromatic ring systems can optionally be substituted with a designated number of substituents, as described herein.

An “aralkyl group” (arylalkyl) is an alkyl group substituted with an aromatic group, preferably a phenyl group. A preferred aralkyl group is a benzyl group. Suitable aromatic groups are described herein and suitable alkyl groups are described herein. An aralkyl group can optionally be substituted, and suitable substituents for an aralkyl group (substituted on the aryl, alkyl or both moieties) are described herein.

As used herein, many moieties or groups are referred to as being either “substituted or unsubstituted”. When a moiety is referred to as substituted, it denotes that any portion of the moiety that is known to one skilled in the art as being available for substitution can be substituted. For example, the substitutable group can be a hydrogen atom which is replaced with a group other than hydrogen (i.e., a substituent group). Multiple substituent groups can be present. When multiple substituents are present, the substituents can be the same or different and substitution can be at any of the substitutable sites on the group or moiety. Such means for substitution are well-known in the art. For purposes of exemplification, which should not be construed as limiting the scope of this invention, some examples of groups that are substituents are: alkyl groups (which can also be substituted, such as CF₃), alkoxy groups (which can be substituted, such as OCF₃), a halogen or halo group (F, Cl, Br, I), hydroxy, nitro, oxo, —CN, —COH, —COOH, amino, N-alkylamino or N,N-dialkylamino (in which the alkyl groups can also be substituted), esters (—C(O)—OR, where R can be a group such as alkyl, aryl, etc., which can be substituted), aryl (most preferred is phenyl, which can be substituted) and arylalkyl (which can be substituted).

N-oxide refers a functionality wherein an oxygen atom is bonded to the nitrogen of a tertiary amine.

### Stereochemistry

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture.

Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When compounds of the present invention contain one chiral center, the compounds exist in two enantiomeric forms and the present invention includes either or both enantiomers and mixtures of enantiomers, such as the specific 50:50 mixture referred to as a racemic mixture. The enantiomers can be resolved by methods known to those skilled in the art, for example by formation of diastereoisomeric salts which may be separated, for example, by crystallization (See, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least about 99% enantiomeric excess.

When a compound of the present invention has two or more chiral carbons, it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

### Pharmacological Methods

### Acute Models

### Dilute Acetic Acid Model and Protamine Sulfate/Physiological Urinary Potassium Model

The acute models described below provide methods for evaluating active agents in the treatment of overactive bladder. Briefly, the models provide a method for reducing the bladder capacity of test animals by infusing either protamine sulfate and potassium chloride (See, Chuang, Y. C. et al., Urology 61(3): 664-670 (2003)) or dilute acetic acid (See, Sasaki, K. et al., J. Urol. 168(3): 1259-1264 (2002)) into the bladder. The infusates cause irritation of the bladder and a reduction in bladder capacity by selectively activating bladder afferent fibers, such as C-fiber afferents. Following irritation of the bladder, an active agent (drug) can be administered and the ability of the active agent to reverse (partially or totally) the reduction in bladder capacity resulting from the irritation, can be determined. Substances which reverse the reduction in bladder capacity can be used in the treatment of overactive bladder.

Animal Preparation for Acute Models:

Female rats (250-275 g BW) are anesthetized with urethane (1.2 g/kg) and a saline-filled jugular catheter (PE-50) is inserted for intravenous drug administration and a heparinized (100 units/ml) saline-filled carotid catheter (PE-50) is inserted for blood pressure monitoring. Via a midline abdominal incision from xyphoid to navel, a PE-50 catheter is inserted into the bladder dome for bladder filling and pressure recording. The abdominal cavity is moistened with saline and closed by covering with a thin plastic sheet in order to maintain access to the bladder for filling cystometry emptying purposes. Fine silver or stainless steel wire electrodes are inserted into the external urethral sphincter (EUS) percutaneously for electromyography (EMG).

Dilute Acetic Acid Model:

Saline and all subsequent infusates are continuously infused at a rate of about 0.055 ml/min via the bladder filling catheter for 30-60 minutes to obtain a baseline of lower urinary tract activity (continuous cystometry; CMG). Bladder pressure traces act as direct measures of bladder and urethral outlet activity, and EUS-EMG phasic firing and voiding act as indirect measures of lower urinary tract activity during continuous transvesical cystometry. Following the control period, a 0.25% acetic acid solution in saline (AA) is infused into the bladder to induce bladder irritation. Following 30 minutes of AA infusion, 3 vehicle injections are made at 20 minute intervals to determine vehicle effects, if any. Subsequently, increasing doses of a selected active agent are administered intravenously at 30 minute intervals in order to construct a cumulative dose-response relationship. At the end of the control saline cystometry period, the third vehicle injection, and 20 minutes following each subsequent treatment, the infusion pump is stopped, the bladder is emptied by fluid withdrawal via the infusion catheter and a single filling cystometrogram is performed at the same flow rate in order to determine changes in bladder capacity caused by the irritation protocol and subsequent drug administration.

Protamine Sulfate/Physiological Urinary Potassium Model:

Saline and all subsequent infusates are continuously infused at a rate of about 0.055 ml/min via the bladder filling catheter for about 30-60 minutes to obtain a baseline of lower urinary tract activity (continuous cystometry; CMG). Bladder pressure traces act as direct measures of bladder and urethral outlet activity, and EUS-EMG phasic firing and voiding act as indirect measures of lower urinary tract activity during continuous transvesical cystometry. Following the control period, a 10 mg/mL protamine sulfate (PS) in saline solution is infused for about 30 minutes in order to permeabilize the urothelial diffusion barrier. After PS treatment, the infusate is switched to 300 mM KCl in saline to induce bladder irritation. Once a stable level of lower urinary tract hyperactivity is established (20-30 minutes), 3 vehicle injections are made at about 30 minute intervals to assess the effects of the vehicle. Subsequently, increasing doses of a selected active agent are administered intravenously at about 30 minute intervals in order to construct a cumulative dose-response relationship. At the end of the control saline cystometry period, the third vehicle injection, and 20 minutes following each subsequent treatment, the infusion pump is stopped, the bladder is emptied by fluid withdrawal via the infusion catheter and a single filling cystometrogram is performed at the same flow rate in order to determine changes in bladder capacity caused by the irritation protocol and subsequent drug administration.

### Chronic Model

### Chronic Spinal Cord Injury Model

The following is a model of neurogenic bladder, in which C-fiber afferents are chronically activated as a result of spinal cord injury (See, Yoshiyama, M. et al., Urology 54(5): 929-933 (1999)). Following spinal cord injury an active agent (drug) can be administered and the ability of the active agent to reverse (partially or totally) the reduction in bladder capacity resulting from spinal cord injury can be determined. Substances which reverse the reduction in bladder capacity can be used in the treatment of overactive bladder, for example, neurogenic bladder.

Animal Preparation for Chronic Model:

Female Sprague-Dawley rats (Charles River, 250-300 g) are anesthetized with isofluorane (4%) and a laminectomy is performed at the T9-10 spinal level. The spinal cord is transected and the intervening space filled with Gelfoam. The overlying muscle layers and skin are sequentially closed with suture, and the animals are treated with antibiotic (100 mg/kg ampicillin s.c.). Residual urine is expressed prior to returning the animals to their home cages, and thereafter 3 times daily until terminal experimentation four weeks later. On the day of the experiment, the animals are anesthetized with isofluorane (4%) and a jugular catheter (PE10) is inserted for access to the systemic circulation and tunneled subcutaneously to exit through the midscapular region. Via a midline abdominal incision, a PE50 catheter with a fire-flared tip is inserted into the dome of the bladder through a small cystotomy and secured by ligation for bladder filling and pressure recording. Small diameter (75 μm) stainless steel wires are inserted percutaneously into the external urethral sphincter (EUS) for electromyography (EMG). The abdominal wall and the overlying skin of the neck and abdomen are closed with suture and the animal is mounted in a Ballman-type restraint cage. A water bottle is positioned within easy reach of the animal's mouth for ad libitum access to water. The bladder catheter is hooked up to the perfusion pump and pressure transducer, and the EUS-EMG electrodes to their amplifier. Following a 30 minute recovery from anesthesia and acclimatization, normal saline is infused at a constant rate (0.100-0.150 ml/min) for control cystometric recording.

Chronic Spinal Cord Injury Model:

Following a 60-90 minute control period of normal saline infusion (0.100-0.150 ml/min) to collect baseline continuous open cystometric data, the pump is turned off, the bladder is emptied, the pump turned back on, and bladder capacity is estimated by a filling cystometrogram. At 3×20-30 minute intervals, vehicle is administered intravenously in order to ascertain vehicle effects on bladder activity. Following the third vehicle control, bladder capacity is again estimated as described above. Subsequently, a cumulative dose-response is performed with the agent of choice. Bladder capacity is measured 20 minutes following each dose.

### EXEMPLIFICATION

The present invention will now be illustrated by the following Example, which is not intended to be limiting in any way.

### Treatment of Overactive Bladder using Tramadol, Gabapentin and a Combination Thereof

The effect of the administration of the α₂δ subunit calcium channel ligand, gabapentin, the substituted aminomethyl-phenyl-cyclohexane derivative, tramadol, and a combination of gabapentin and tramadol to reverse the reduction in bladder capacity using the Dilute Acetic Acid Model, was assessed.

Materials and Methods

Urethane anesthetized (1.2 g/kg) normal female rats were utilized in this study. Groups of rats were treated with tramadol alone (n=4), gabapentin alone (n=11), and respective dose-matched combinations of tramadol and gabapentin (n=6). Cumulative dose-response protocols were utilized with half log increments for all studies.

Drugs and Preparation

Drugs were dissolved in normal saline at 3, 10 and 30 mg/ml for tramadol and 30, 100 and 300 mg/ml for gabapentin. In these studies, individual doses and combinations may be subsequently referred to as Low, Mid and High. Animals were dosed by volume of injection=body weight in kg.

Dilute Acetic Acid Model

Female rats (250-300 g BW, n=14) were anesthetized with urethane (1.2 g/kg) and a saline-filled jugular catheter (PE-10) was inserted for access to the systemic circulation. A PE-50 catheter having a flared tip was inserted into the bladder dome via a midline lower abdominal incision and secured by ligation for bladder filling and pressure recording. The abdominal cavity was moistened with saline and closed by covering with a thin plastic sheet in order to maintain access to the bladder for filling cystometry emptying purposes. Fine silver or stainless steel wire electrodes were inserted into the external urethral sphincter (EUS) percutaneously for electromyography (EMG). Animals were positioned on a heating pad which maintained body temperature at 37° C.

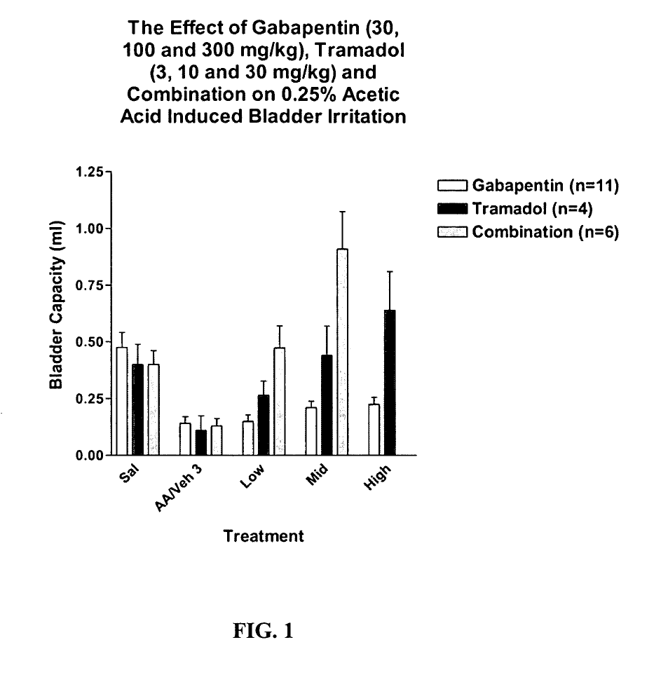

Saline (and all subsequent infusates) were continuously infused at a rate of about 0.055 mL/min via the bladder filling catheter for 30-60 minutes to obtain a baseline of lower urinary tract activity (continuous cystometry; CMG). At the end of the control saline cystometry period, the infusion pump was stopped, the bladder was emptied by fluid withdrawal via the infusion catheter and a single filling cystometrogram was performed at the same flow rate in order to measure bladder capacity. Bladder pressure traces act as direct measures of bladder and urethral outlet activity, and EUS-EMG phasic firing and voiding act as indirect measures of lower urinary tract activity during continuous transvesical cystometry. Following the control period, a 0.25% acetic acid solution in saline (AA) was infused into the bladder to induce bladder irritation. Following 30 minutes of AA infusion, 3 vehicle injections (saline, 1 mL/kg dose) were made at 20 minute intervals to determine vehicle effects, if any on the acetic acid irritation of the bladder and to achieve a stable level of irritation with this dilute acetic acid solution. Following injection of the third vehicle control, bladder capacity was again estimated as described above. Selected doses of Tramadol, Gabapentin and a combination of Tramadol and Gabapentin were administered intravenously and bladder capacity was again measured 20 minutes following administration. The results are set forth graphically in FIGS. 1-3 and details of the dosing regimen are set forth in the Table.

TABLE

| AGENT | DOSE 1 (LOW) | DOSE 2 (MID) | DOSE 3 (HIGH) |
|---|---|---|---|
| Gabapentin | 30 mg/kg | 100 mg/kg | 300 mg/kg |
| (n = 11) | | | |
| Tramadol (n = 4) | 3 mg/kg | 10 mg/kg | 30 mg/kg |
| COMBINATION: | LOW | MD | HIGH |
| Gabapentin and | 30 mg/kg | 100 mg/kg | 300 mg/kg |
| Tramadol (n = 6) | 3 mg/kg | 10 mg/kg | 30 mg/kg |

Data Analysis

Bladder capacity data for each animal were normalized to “% Recovery from Irritation,” and this index was used as the measure of efficacy. Data from experiments in which each of the drugs were administered alone were utilized to create theoretical populations of additive effects for each dose (low, mid and high), and these were compared by one-tailed t-test (individual dose comparisons) and by 2-Way ANOVA (across doses) to the actual combination drug data. The means and standard deviations of each individual treatment's “dose-matched” (low, middle, and high) responses were added together to estimate the mean and standard deviation of the theoretical additive populations for which to compare to the actual data obtained from the combination experiments. The theoretical additive effect population N=(N_{tramadol}+N_{gabapentin})−1. P<0.050 was considered significant.

Results

The effect of cumulative increasing doses of tramadol (n=4), gabapentin (n=11) and their matched combinations (e.g., Low Dose for the combination was 30 mg/kg gabapentin and 3 mg/kg tramadol; n=6) on bladder capacity is depicted in FIG. 1. Data are presented as Mean±SEM. The high dose combination resulted in respiratory depression resulting ultimately in death, and data from this combination dose are therefore not included.

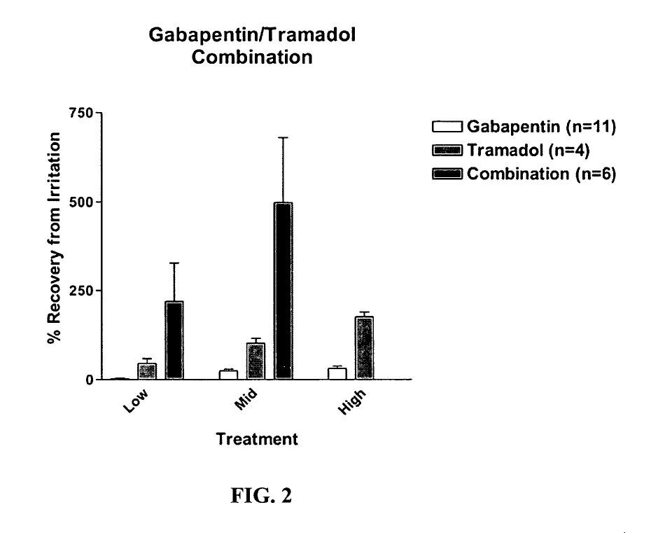

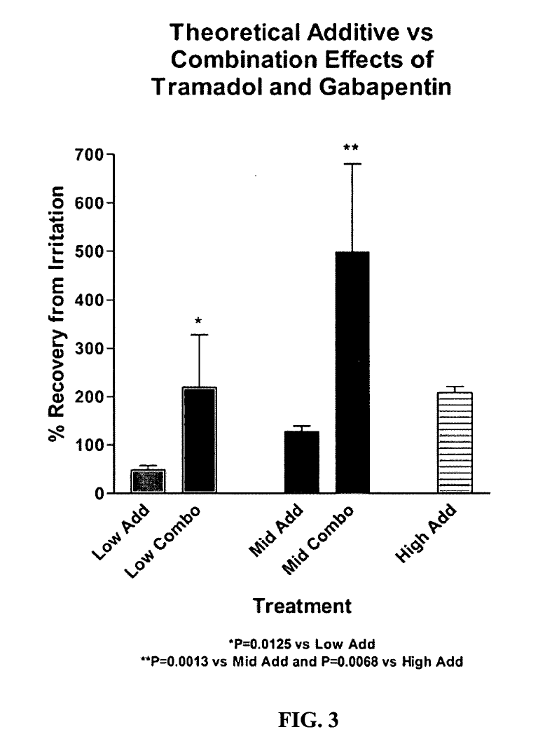

The effect of cumulative increasing doses of tramadol (n=4), gabapentin (n=11) and their matched combinations (e.g, Low Dose for the combination was 30 mg/kg gabapentin and 3 mg/kg tramadol; n=6) on bladder capacity (normalized to % Recovery from Irritation) is depicted in FIG. 2. The theoretical additive results are compared to actual combination results in FIG. 3. Note that the combination of drugs produced a greater than additive effect at the Low (P=0.0125) and Mid doses (P=0.0013), on reduction in bladder capacity caused by continuous intravesical exposure to dilute acetic acid. Moreover, the Mid dose combination effect was significantly greater than the theoretical additive effect for the high dose. Data are presented as Mean±SEM.

Conclusions

The ability of an α₂δ subunit calcium channel ligand in combination with a substituted aminomethyl-phenyl-cyclohexane derivative to produce a dramatic reversal in acetic acid irritation-induced reduction in bladder capacity strongly indicates efficacy in mammalian forms of lower urinary tract disorders and associated irritative symptoms in normal and spinal cord injured patients. Furthermore, the combination of an α₂δ subunit calcium channel ligand and a substituted aminomethyl-phenyl-cyclohexane derivative produced a synergistic effect that was greater than what would be expected if the effects were simply additive, and also demonstrated efficacy using amounts of the individual agents that are much lower than would be expected to produce an effect if the agents were administered singly.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating at least one symptom of a lower urinary tract disorder in a subject in need of treatment, wherein the symptom is selected from the group consisting of urinary frequency, urinary urgency, urinary urge incontinence, nocturia and enuresis, comprising administering to said subject:
a) a first amount of an α₂δ subunit calcium channel ligand; and
b) a second amount of a substituted aminomethyl-phenyl-cyclohexane derivative,
wherein the first and second amounts together comprise a therapeutically effective amount.

2. The method of claim 1, wherein the lower urinary tract disorder is selected from the group consisting of overactive bladder, interstitial cystitis, prostatitis, prostadynia and benign prostatic hyperplasia.

3. The method of claim 2, wherein the lower urinary tract disorder is overactive bladder.

4. The method of claim 2, wherein the lower urinary tract disorder is interstitial cystitis.

5. The method of claim 1, wherein the subject is a human.

6. The method of claim 1, wherein the α₂δ subunit calcium channel ligand is a GABA analog.

7. The method of claim 6, wherein the GABA analog is selected from the group consisting of gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and combinations thereof.

8. The method of claim 7, wherein the GABA analog is gabapentin, pregabalin or a combination thereof.

9. The method of claim 1, wherein the substituted aminomethyl-phenyl-cyclohexane derivative is represented by structural Formula I: 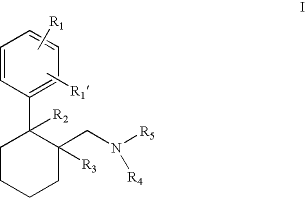
and enantiomers and mixtures thereof wherein:
R₁ and R₁′ are independently hydrogen, an aliphatic group, an aryl group, an arylalkyl group, a halogen, —CN, —OR₆, —SR₆, —NR₆R₆, —OC(O)R₆, —C(O)OR₆, —C(O)R₆ or —C(O)NR₆R₆;
R₂ is hydrogen, halogen, —OR₇ or —OC(O)R₇;
R₃ is hydrogen or an aliphatic group;
or R₂ and R₃ together form a double bond;
R₄ and R₅ are independently hydrogen, an aliphatic group, an aryl group or an arylalkyl group;
R₆ is hydrogen, an aliphatic group, an aryl group or an arylalkyl group;
R₇ is hydrogen, an aliphatic group, an aryl group or an arylalkyl group;
or pharmaceutically acceptable salts, solvates or hydrates thereof.

10. The method of claim 9, wherein R₂ is —OH.

11. The method of claim 10, wherein R₁′ is H and R₁ is —OCH₃.

12. The method of claim 11, wherein, —OCH₃ is substituted at the meta position of the phenyl ring.

13. The method of claim 9, wherein the α₂δ subunit calcium channel ligand is a GABA analog.

14. The method claim 13, wherein the GABA analog is selected from the group consisting of gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and a combination thereof.

15. The method of claim 14, wherein the GABA analog is gabapentin, pregabalin or a combination thereof.

16. The method of claim 1, wherein the substituted aminomethyl-phenyl-cyclohexane derivative is represented by structural Formula II: 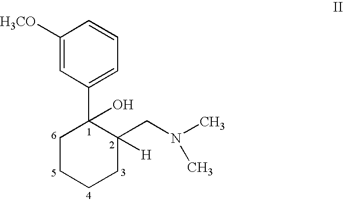
and enantiomers and mixtures thereof or pharmaceutically acceptable salts, solvates or hydrates thereof.

17. The method of claim 16, wherein the compound of Formula II is a mixture of the following enantiomers:

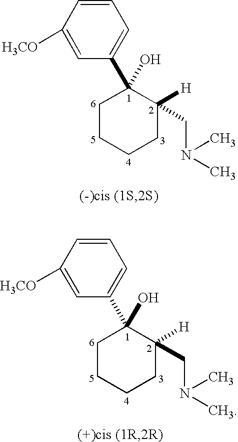

18. The method of claim 17, wherein the mixture is a racemic mixture.

19. The method of claim 17, wherein the compound of Formula II is the (+)cis enantiomer.

20. The method of claim 16, wherein the α₂δ subunit calcium channel ligand is a GABA analog.

21. The method claim 20, wherein the GABA analog is selected from the group consisting of gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and a combination thereof.

22. The method of claim 21, wherein the GABA analog is gabapentin, pregabalin or a combination thereof.

23. A method of treating at least one symptom of a lower urinary tract disorder in a subject in need of treatment, wherein the symptom is selected from the group consisting of urinary frequency, urinary urgency, urinary urge incontinence, nocturia and enuresis, comprising administering to said subject:
a) a first amount of a GABA analog selected from the group consisting of:
gabapentin, pregabalin or a combination thereof; and
b) a second amount of tramadol hydrochloride,
wherein the first and second amounts together comprise a therapeutically effective amount.

24. The method of claim 23, wherein the lower urinary tract disorder is selected from the group consisting of overactive bladder, interstitial cystitis, prostatitis, prostadynia and benign prostatic hyperplasia.

25. The method of claim 24, wherein the lower urinary tract disorder is overactive bladder.

26. The method of claim 24, wherein the lower urinary tract disorder is interstitial cystitis.

27. The method of claim 23, wherein the subject is a human.

28. The method of claim 23, wherein the GABA analog is gabapentin.

29. The method of claim 28 wherein the therapeutically effective amount provides a synergistic effect.

30. A method of treating at least one symptom of a lower urinary tract disorder in a subject in need of treatment, wherein the symptom is selected from the group consisting of urinary frequency, urinary urgency, urinary urge incontinence, nocturia and enuresis, comprising administering to said subject:
a) a first amount of an α₂δ subunit calcium channel ligand; and
b) a second amount of a substituted aminomethyl-phenyl-cyclohexane derivative represented by structural Formula III and enantiomers and mixtures thereof: 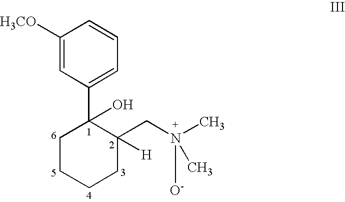
or pharmaceutically acceptable salts, solvates and hydrates thereof, wherein the first and second amounts together comprise a therapeutically effective amount.

31. The method of claim 30, wherein the compound of Formula III is a mixture of the following enantiomers:

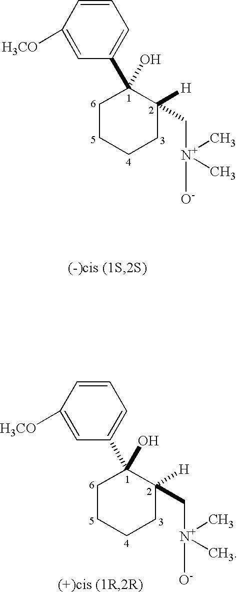

32. The method of claim 31, wherein the mixture is a racemic mixture.

33. The method of claim 31, wherein the compound of Formula III is the (+)cis enantiomer.

34. The method of claim 30, wherein the lower urinary tract disorder is selected from the group consisting of overactive bladder, interstitial cystitis, prostatitis, prostadynia and benign prostatic hyperplasia.

35. The method of claim 34, wherein the lower urinary tract disorder is overactive bladder.

36. The method of claim 34, wherein the lower urinary tract disorder is interstitial cystitis.

37. The method of claim 30, wherein the subject is a human.

38. The method of claim 30, wherein the α₂δ subunit calcium channel ligand is a GABA analog.

39. The method of claim 38, wherein the GABA analog is selected from the group consisting of gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and a combination thereof.

40. The method of claim 39, wherein the GABA analog is gabapentin, pregabalin or a combination thereof.

41. A method of treating at least one symptom of a lower urinary tract disorder in a subject in need of treatment, wherein the symptom is selected from the group consisting of urinary frequency, urinary urgency, urinary urge incontinence, nocturia and enuresis, comprising administering to said subject:
a) a first amount of an α₂δ subunit calcium channel ligand; and
b) a second amount of a substituted aminomethyl-phenyl-cyclohexane derivative selected from the group consisting of (+/−)O-desmethyltramadol, (+)O-desmethyltramadol, (−)O-desmethyltramadol, (+/−)O-desmethyl-N-mono-desmethyl-tramadol, (+)O-desmethyl-N-mono-desmethyl-tramadol, (−) O-desmethyl-N-mono-desmethyl-tramadol and a combination thereof.

42. The method of claim 41, wherein the lower urinary tract disorder is selected from the group consisting of overactive bladder, interstitial cystitis, prostatitis, prostadynia and benign prostatic hyperplasia.

43. The method of claim 42, wherein the lower urinary tract disorder is overactive bladder.

44. The method of claim 42, wherein the lower urinary tract disorder is interstitial cystitis.

45. The method of claim 41, wherein the subject is a human.

46. The method of claim 41, wherein the α₂δ subunit calcium channel ligand is a GABA analog.

47. The method of claim 46, wherein the GABA analog is selected from the group consisting of gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and a combination thereof.

48. The method of claim 47, wherein the GABA analog is gabapentin, pregabalin or a combination thereof.

49. The method of claim 1, wherein the substituted aminomethyl-phenyl-cyclohexane derivative is represented by structural Formula V: 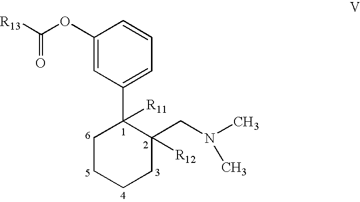
and enantiomers and mixtures thereof wherein:
R₁₁ is —OH;
R₁₂ is hydrogen or R₁₁ and R₁₂ together form a double bond:
R₁₃ is an aryl group selected from the group consisting of: 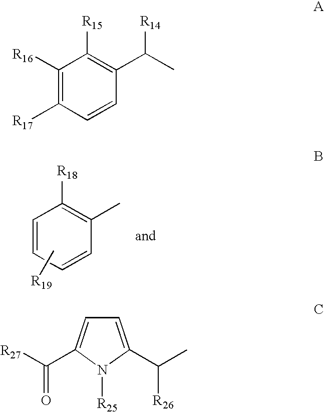
R₁₄ is hydrogen or an alkyl group;
R₁₅ is hydrogen, —NH₂, —NHR₂₀ or —OR₂₀;
R₁₆ is hydrogen, —COR₂₀, —OR₂₀ or halogen;
R₁₇ is hydrogen, an alkyl group, —O-alkenyl, a phenyl group or R₁₆ and R₁₇ are —CH═CR₂₁—CR₂₂═CH—, forming an unsubstituted or substituted with R₂₁ or R₂₂ condensed aromatic ring;
R₁₈ is hydrogen, —COR₂₃, —OR₂₄ or a halogen;
R₁₉ is hydrogen, halogen, an alkyl group, —O-alkyl, —NO₂ or an aryl group;
R₂₀ is a phenyl group optionally substituted by one or more of the following: halogen, —NO₂, an alkyl group, an alkenyl group, —OH or —NH₂;
R₂₁ and R₂₂ are independently hydrogen or —O-alkyl;
R₂₃ is a phenyl group optionally substituted by one or more of the following: halogen, —NO₂, an alkyl group, an alkenyl group, —OH or —NH₂;
R₂₄ is hydrogen, —CO-alkyl (preferably methyl) or a phenyl group optionally substituted by one or more of the following: halogen, —NO₂, an alkyl group, an alkenyl group, —OH or —NH₂;
R₂₅ and R₂₆ are independently hydrogen, an alkyl group or form a —CH₂—CH₂— group;
R₂₇ is a phenyl group optionally substituted by one or more of the following: halogen, —NO₂, an alkyl group, an alkenyl group, —OH or —NH₂;
or pharmaceutically acceptable salts, solvates or hydrates thereof.

50. The method of claim 49, wherein for the compound of Formula V, R₁₁ is —OH, R₁₂ is H and R₁₃ is: 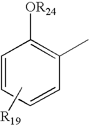
wherein:
R₂₄ is hydrogen or —COCH₃;
R₁₉ is halogen, an alkyl group, —O-alkyl or —NO₂.

51. The method of claim 50, wherein R₁₉ is —O-alkyl.

52. The method of claim 51, wherein R₁₉ is —OCH₃.

53. The method of claim 50, wherein R₁₉ is an alkyl group.

54. The method of claim 53, wherein the R₁₉ is a substituted alkyl group.

55. The method of claim 54, wherein the substituted alkyl group is —CF₃.

56. The method of claim 49, wherein the α₂δ subunit calcium channel ligand is a GABA analog.

57. The method claim 56, wherein the GABA analog is selected from the group consisting of gabapentin, pregabalin, cis-(1S,3R)-(1-(aminomethyl)-3-methylcyclohexane)acetic acid, cis-(1R,3S)-(1-(aminomethyl)-3-methylcyclohexane)acetic acid, 1α,3α,5α-(1-aminomethyl)-(3,5-dimethylcyclohexane)acetic acid, (9-(aminomethyl)bicyclo[3.3.1]non-9-yl)acetic acid, (7-(aminomethyl)bicyclo[2.2.1]hept-7-yl)acetic acid and a combination thereof.

58. The method of claim 57, wherein the GABA analog is gabapentin, pregabalin or a combination thereof.

59. A kit comprising a sub-therapeutic dose of a compound which is an α₂δ subunit calcium channel ligand, instructions for use with a compound which is a substituted aminomethyl-phenyl-cyclohexane derivative and optionally a device for administering the compounds.

60. A kit comprising a sub-therapeutic dose of a compound which is a substituted aminomethyl-phenyl-cyclohexane derivative, instructions for use with a compound which is an α₂δ subunit calcium channel ligand and optionally a device for administering the compounds.

61. A kit comprising a first compound which is an α₂δ subunit calcium channel ligand, a second compound which is a substituted aminomethyl-phenyl-cyclohexane derivative and instructions for administering the first and second compounds and optionally a device for administering the compounds, wherein at least one of said first or second compound is present in a sub-therapeutic dose.
